SEQUENCE LISTING (3 sequences)

SEQ ID NO: 1 - DNA, 93 bp, Artificial
  aptamer library transcription template ARC254
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, Artificial
  aptamer library transcription template ARC 255
catgcatcgcgactgactagccgnnnnnnnnnnnnnnnnnnnnnnnnnnnnnngtagaacgttctctcctctccctatagtgagtcgtatta

SEQ ID NO: 3 - DNA, 92 bp, Artificial
  aptamer library transcription template ARC 256
catcgatcgatcgatcgacagcgnnnnnnnnnnnnnnnnnnnnnnnnnnnnnngtagaacgttctctcctctccctatagtgagtcgtatta

(19) United States
(12) Patent Application Publication
(10) Pub. No.: US 2004/0249130 A1
(43) Pub. Date: Dec. 9, 2004

(54) Aptamer-toxin molecules and methods for using same

(76) Inventors: Martin Stanton, Concord, MA (US); Markus Kurz, Newton, MA (US); Charles Wilson, Concord, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/826,077

(22) Filed: Apr. 15, 2004

Related U.S. Application Data
(63) Continuation-in-part of application No. 10/600,007, filed on Jun. 18, 2003.
(60) Provisional application No. 60/390,042, filed on Jun. 18, 2002.

(51) Int. Cl. C12Q 1/68; C07K 1/00; C07K 17/00; C07K 14/00

(52) U.S. Cl. 530/350

(57) ABSTRACT

Materials and methods are provided to prepare therapeutic conjugates for the treatment of proliferative diseases. The therapeutic conjugates of the invention comprise a targeting moiety conjugated to a therapeutic moiety. The therapeutic moiety of the conjugates of the present invention have a cytotoxic effect and are useful in the treatment of proliferative diseases.

### REFERENCE TO RELATED APPLICATIONS

This application is a continuation-in-part of U.S. patent application Ser. No. 10/600,007, filed on Jun. 18, 2003, which claims priority to and is related to U.S. Provisional Application Ser. No. 60/390,042, filed Jun. 18, 2002, each of which is incorporated by reference herein.

### FIELD OF THE INVENTION

The invention relates generally to the field of nucleic acids and more particularly to compositions and methods for delivering cytotoxic agents to cells by linking a nucleic acid aptamer to cytotoxic agents and delivering the aptamer-toxin conjugate to a target. Similarly, a nucleic acid sensor molecule (NASM) can be linked to a toxin and the NASM-toxin conjugate delivered to a target.

### BACKGROUND OF THE INVENTION

Aptamers are nucleic acid molecules having specific binding affinity to non-nucleic acid or nucleic acid molecules through interactions other than classic Watson-Crick base pairing. Aptamers are described e.g., in U.S. Pat. Nos. 5,475,096; 5,270,163; 5,589,332; 5,589,332; and 5,741,679, each of which is incorporated in its entirety by reference herein.

Aptamers, like peptides generated by phage display or monoclonal antibodies (MAbs), are capable of specifically binding to selected targets and, through binding, blocking their targets' ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes.

Aptamers have a number of desirable characteristics for use as therapeutics including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial therapeutic leads. In vitro selection allows the specificity and affinity of the aptamer to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptamer: 10-50 KD; antibody: 150 KD), a weekly dose of aptamer may be delivered by injection in a volume of less than 0.5 ml. Aptamer bioavailability via subcutaneous administration is >80% in monkey studies (Tucker et al., J. Chromatography B. 732: 203-212, 1999).

4) Scalability and cost. Therapeutic aptamers are chemically synthesized and consequently can be readily scaled as needed to meet production demand. Whereas difficulties in scaling production are currently limiting the availability of some biologics (e.g., Enbrel, Remicade) and the capital cost of a large-scale protein production plant is enormous (e.g., $500 MM, Immunex), a single large-scale synthesizer can produce upwards of 100 kg oligonucleotide per year and requires a relatively modest initial investment (e.g., <$10 MM, Avecia). The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Cytotoxic agents are molecules that have lethal or growth inhibiting effects on cells. Cytotoxic or chemotherapeutics agents can be classified as tubulin stabilizers or destabilizers, anti-metabolites, purine synthesis inhibitors, nucleoside analogs, and DNA alkylating or other DNA modifying agents. Such agents have been used as therapeutics in proliferative diseases such as cancer, solid tumors, inflammation diseases, overactive scarring disorders, and autoimmune diseases such as lupus. Because of their cytotoxic effect these chemotherapeutic agents tend to also affect or inhibit healthy or non-target cells leading to undesirable morbidity or side effects in subjects or patients being treated.

There is a need for delivery of cytotoxic or therapeutic agents to treat proliferative diseases that maximize cytotoxicity to diseased malignant cells or target cells without collateral cytotoxicity to healthy or normal cells or surrounding tissue.

The materials and methods of the present invention provide a target specific therapeutic agent-aptamer complex that increases the effectiveness of cytotoxic agents or therapeutics and minimizes damage to non-target cells. The aptamer-toxin conjugates and methods of the present invention meet these and other needs.

### BRIEF DESCRIPTION OF THE DRAWINGS

FIG. 1 is a schematic representation of the in vitro aptamer selection (SELEX™) process from pools of random sequence oligonucleotides.

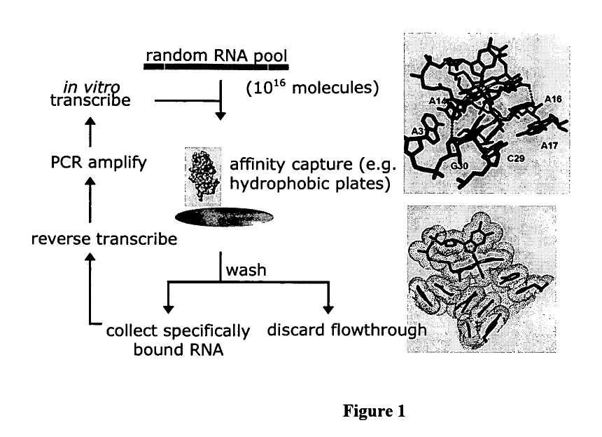

FIG. 2 is a schematic diagram in which the oligonucleotide population is screened for a nucleic acid sensor molecule which comprises a target molecule activatable ligase activity.

FIG. 3 is an illustration depicting the hammerhead nucleic acid sensor molecule selection methodology.

FIG. 4 is an illustration depicting an aptamer bearing four free reactive amines produced by two rounds of coupling with a 5′-symmetric doubler followed by amine capping.

FIG. 5 is an illustration depicting various strategies for synthesis of high molecular weight PEG-nucleic acid conjugates.

### SUMMARY OF THE INVENTION

The specificity of aptamers allows them to be used as molecular “chaperones” to increase the specificity of another molecule to a given target by linking said molecule to an aptamer with high binding affinity to a target.

In one embodiment, a cytotoxic agent or toxin is linked to an aptamer, forming a toxin-aptamer conjugate molecule that increases the specificity of the cytotoxic agent moiety to a given specific target. In one embodiment of the toxin-aptamer conjugate, the toxin or cytotoxic agent is a chemotoxin.

In one embodiment, the aptamer-toxin conjugate is used as a chemotherapeutic agent in the treatment of proliferative diseases including, but not limited to, inflammation disorders, scarring, solid tumor cancers, autoimmune disorders, including lupus for instance.

In another embodiment, the toxin conjugate is a protein toxin. In one embodiment, the protein is an antibody or antibody fraction. In another embodiment the toxin is a protein having binding specificity and affinity for another molecule.

In another embodiment, the toxin is a nucleic acid toxin.

In another embodiment, the chemotoxin conjugate is a small molecule therapeutic agent including but not limited to tubulin stabilizers/destabilizers, anti-metabolites, purine synthesis inhibitors, nucleoside analogs, and DNA alkylating or other DNA-modifying agents, including for instance doxorubicin.

In another embodiment, the chemotoxin conjugate includes but is not limited to calichomycin, doxorubicin, taxol, methotrexate, gencitadine, AraC (cytarabine), vinblastin, daunorubicin.

In another embodiment, the toxic agent is a radioisotope.

In another embodiment, the targets for the toxin-aptamer conjugate are cell surface receptors, including but not limited to receptor tyrosine kinases, EGFR, her2 new, PSMA, and Muc1.

The specificity of NASMs allows them to be used as molecular “chaperones” to increase the specificity of another molecule to a given target by linking said molecule to a NASM which recognizes a target with high specificity.

In one embodiment, a cytotoxic agent or toxin is linked to a NASM, forming a toxin-NASM conjugate molecule that increases the specificity of the cytotoxic agent moiety to a given specific target. In one embodiment of the toxin-NASM conjugate, the toxin or cytotoxic agent is a chemotoxin.

In one embodiment, the NASM-toxin conjugate is used as a chemotherapeutic agent in the treatment of proliferative diseases including, but not limited to, inflammation disorders, scarring, solid tumor cancers, autoimmune disorders, including lupus for instance.

In another embodiment, the toxin conjugate is a protein toxin. In one embodiment, the protein is an antibody or antibody fraction. In another embodiment the toxin is a protein having binding specificity and affinity for another molecule.

In another embodiment, the toxin is a nucleic acid toxin.

In another embodiment, the chemotoxin conjugate is a small molecule therapeutic agent including but not limited to tubulin stabilizers/destabilizers, anti-metabolites, purine synthesis inhibitors, nucleoside analogs, and DNA alkylating or other DNA-modifying agents, including for instance doxorubicin.

In another embodiment, the chemotoxin conjugate includes but is not limited to calichomycin, doxorubicin, taxol, methotrexate, gencitadine, AraC (cytarabine), vinblastin, daunorubicin.

In another embodiment, the toxic agent is a radioisotope.

In another embodiment, the targets for the toxin-NASMs conjugate are cell surface receptors, including but not limited to receptor tyrosine kinases, EGFR, her2 new, PSMA, and Muc1.

The invention also provides aptamer-drug conjugates that include one or more aptamers and a drug linked by a linker and having the formula: (aptamer)ₙ—linker—(drug)ₘ, wherein n is between 1 and 10 and m is between 0 and 20. In one embodiment, the one or more aptamers is a tumor-cell targeting aptamer. These tumor-cell targeting aptamers are specific for a target such as PSMA, PSCA, e-selectin, an ephrin, ephB2, cripto-1, TENB2 (TEMFF2), ERBB2 receptor (HER2), MUC1, CD44v6, CD6, CD19, CD20, CD22, CD23, CD25, CD30, CD33, CD56, IL-2 receptor, HLA-DR10β subunit, EGFRvIII, MN antigen, caveolin-1 and nucleolin.

In one embodiment, the drug is a cytotoxin. For example, the cytotoxin is calicheamicin, a maytansinoid, a vinca alkaloid, a cryptophycin, a tubulysin, dolastatin-10, dolastatin-15, auristatin E, rhizoxin, epothilone B, epithilone D, taxoids or variants thereof. Other suitable cytotoxins include Nac-γ-DMH, Nac-γ-NHS, maytansine, May-NHS, desacetyl vinblastine 3-carboxhydrazide (DAVCH), desacetyl vinblastine 4-O-succinate (DAVS), cryptophycin-52, and crypthophycin-52-amine (Cryp-NH2).

In one embodiment, the linker has one or more nucleophilic moieties, one or more electrophilic moieties or combinations thereof. Other suitable linkers include a Boc-protected amine, a Boc-protected amine on a heterobifunctional linker, a nucleophilic dendrimer, an electrophilic dendrimer or an electrophilic comb polymer. For example, the linker is Boc-NH2-PEG-NHS, an erythritol dendrimer, an octa-polyethylene glycol dendrimer or a comb polymer.

### DETAILED DESCRIPTION OF THE INVENTION

Definitions

As defined herein, “toxin” is a molecule having a deleterious effect on another molecule or living cell, potentially resulting in the ultimate death of the cell.

As defined herein, “nucleic acid” means either DNA, RNA, single-stranded or double-stranded, and any chemical modifications thereof. Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

As defined herein, “oligonucleotide” is used interchangeably with the term “nucleic acid” and includes RNA or DNA (or RNA/DNA) sequences of more than one nucleotide in either single strand or double-stranded form. A “modified oligonucleotide” includes at least one nucleotide residue with any of: an altered internucleotide linkage(s), altered sugar(s), altered base(s), or combinations thereof.

As defined herein, “target” means any compound or molecule of interest for which a nucleic acid ligand exists or can be generated. A target molecule can be naturally occurring or artificially created, including a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc. without limitation.

As defined herein, a nucleic acid sensor molecule which “recognizes a target molecule” is a nucleic acid molecule whose activity is modulated upon binding of a target molecule to the target modulation domain to a greater extent than it is by the binding of any non-target molecule or in the absence of the target molecule. The recognition event between the nucleic acid sensor molecule and the target molecule need not be permanent during the time in which the resulting allosteric modulation occurs. Thus, the recognition event can be transient with respect to the ensuing allosteric modulation (e.g., conformational change) of the nucleic acid sensor molecule.

As defined herein, a molecule which “naturally binds to DNA or RNA” is one which is found within a cell in an organism found in nature.

As defined herein, a “random sequence” or a “randomized sequence” is a segment of a nucleic acid having one or more regions of fully or partially random sequences. A fully random sequence is a sequence in which there is an approximately equal probability of each base (A, T, C, and G) being present at each position in the sequence. In a partially random sequence, instead of a 25% chance that an A, T, C, or G base is present at each position, there are unequal probabilities.

As defined herein, an “aptamer” is a nucleic acid which binds to a non-nucleic acid target molecule or a nucleic acid target through non-Watson-Crick base pairing.

As defined herein, an aptamer nucleic acid molecule which “recognizes a target molecule” is a nucleic acid molecule which specifically binds to a target molecule.

As defined herein, a “nucleic acid sensor molecule” or “NASM” refers to either or both of a catalytic nucleic acid sensor molecule and an optical nucleic acid sensor molecule.

As defined herein, a “nucleic acid ligand” refers to either or both an aptamer or a NASM.

As defined herein, a “catalytic nucleic acid sensor molecule” is a nucleic acid sensor molecule comprising a target modulation domain, a linker region, and a catalytic domain.

As defined herein, an ‘optical nucleic acid sensor molecule” is a catalytic nucleic acid sensor molecule wherein the catalytic domain has been modified to emit an optical signal as a result of and/or in lieu of catalysis by the inclusion of an optical signal generating unit.

As defined herein, a “target modulation domain” (TMD) is the portion of a nucleic acid sensor molecule which recognizes a target molecule. The target modulation domain is also sometimes referred to herein as the “target activation site” or “effector modulation domain”.

As defined herein, a “catalytic domain” is the portion of a nucleic acid sensor molecule possessing catalytic activity which is modulated in response to binding of a target molecule to the target modulation domain.

As defined herein, a “linker region” or “linker domain” is the portion of a nucleic acid sensor molecule by or at which the “target modulation domain” and “catalytic domain” are joined. Linker regions include, but are not limited to, oligonucleotides of varying length, base pairing phosphodiester, phosphothiolate, and other covalent bonds, chemical moieties (e.g., PEG), PNA, formacetal, bismaleimide, disulfide, and other bifunctional linker reagents. The linker domain is also sometimes referred to herein as a “connector” or “stem”.

As defined herein, an “optical signal generating unit” is a portion of a nucleic acid sensor molecule comprising one or more nucleic acid sequences and/or non-nucleic acid molecular entities, which change optical or electrochemical properties or which change the optical or electrochemical properties of molecules in close proximity to them in response to a change in the conformation or the activity of the nucleic acid sensor molecule following recognition of a target molecule by the target modulation domain.

As defined herein, “specificity” refers to the ability of a nucleic acid of the present invention to recognize and discriminate among competing or closely-related targets or ligands. The degree of specificity of a given nucleic acid is not necessarily limited to, or directly correlated with, the binding affinity of a given molecule. For example, hydrophobic interaction between molecule A and molecule B has a high binding affinity, but a low degree of specificity. A nucleic acid that is 100 times more specific for target A relative to target B will preferentially recognize and discriminate for target A 100 times better than it recognizes and discriminates for target B.

As defined herein, “selective” refers to a molecule that has a high degree of specificity for a target molecule.

The invention is based in part on the discovery of compositions that include a nucleic acid moiety linked to a cytotoxic agent. The nucleic acid moiety binds to a desired cell or cell surface marker. The linked cytotoxic agent is thus brought in close proximity of the cell, which allows for the cytotoxic agent to exert its cytotoxic effects on the cell. The use of these aptamer-toxin conjugates allows for the selective delivery of cytotoxic molecules to target cells.

In one aspect, the invention provides an aptamer-toxin conjugate wherein the toxin is a chemotoxin. In some embodiments, the toxin is a protein toxin. In other embodiments, the toxin is a nucleic acid toxin.

In some embodiments, the toxin is attached to the aptamer through covalent bond. If desired, the toxin is attached to an aptamer through a hydrolysable bond, and/or through a bond that can be cleaved through enzymatic activity.

In other embodiments, the toxin is attached to the aptamer through a non-covalent bond.

In some embodiments, the aptamer-toxin conjugate binds to target, thereby delivering toxin to the vicinity of the target. The toxin may interact with the same target, or with a second target in the vicinity of the first target.

In some embodiments, binding to the target results in the translocation of the aptamer and associated toxin. For example, binding to the target results in the translocation of the aptamer and associated toxin across a cell membrane. In some embodiments, binding to target results in the translocation of the aptamer and associated toxin through structures in an organ, tissue or cell.

In some embodiments, the aptamer-toxin conjugate binds to a target, and binding to target results in a change in conformation of the aptamer-toxin. The change in conformation results in a change in activity of the aptamer-toxin.

For example, in some embodiments, binding of the aptamer-toxin conjugate to a target can result in a change in conformation of the aptamer-toxin conjugate, such change resulting in a release of the toxin.

Alternatively, or in addition, binding of the aptamer-toxin conjugate to a target can result in a change in conformation of the aptamer-toxin conjugate, wherein the conformational change results in an activation of the toxin.

In a further embodiment, the aptamer-toxin conjugate binds to a target, where binding to target results in a change in conformation of the aptamer-toxin conjugate, and the change results in inactivation of the toxin.

In various embodiments, an aptamer-toxin conjugate is provided whose half-life is less than, equal to, or greater than, the half-life of the toxin.

Also provided by the invention is a method of generating an aptamer-toxin conjugate that includes attaching a toxin to an aptamer. In some embodiments, the aptamer in the moiety is created using a process termed “Systematic Evolution of Ligands by EXponential enrichment” (the “SELEX process”). The SELEX process is a method for the in vitro evolution of nucleic acid molecules with highly specific binding to target molecules and is described in, e.g., U.S. Pat. No. 5,475,096 entitled “Nucleic Acid Ligands”, and U.S. Pat. No. 5,270,163 (see also WO91/19813) entitled “Nucleic Acid Ligands”.

For example, the invention includes a method of generating an aptamer-toxin conjugate by attaching a toxin to a random pool of nucleic acids and then using the SELEX process to find the optimized aptamer-toxin conjugate from within the random pool. Alternatively, a toxin can be attached to an aptamer post-selection.

In some embodiments, the method of generating an aptamer-toxin conjugate results in a aptamer whose half-life is engineered to match the half life of the toxin. For example, the invention includes a method of generating an aptamer-toxin conjugate where the aptamer half life is engineered to match the half life of the toxin by adjusting the percentage of nuclease resistant bases in the aptamer. In other embodiments, the invention includes a method of generating an aptamer-toxin conjugate where the aptamer half life is engineered to match the half life of the toxin by changing the 5′ and/or 3′ end capping.

Also within the invention is a NASM-toxin conjugate wherein the toxin is a chemotoxin. In some embodiments, the toxin is a protein toxin. In other embodiments, the toxin is a nucleic acid toxin.

In some embodiments, the toxin is attached to the NASM through covalent bond. If desired, the toxin is attached to a NASM through a hydrolysable bond, and/or through a bond that can be cleaved through enzymatic activity.

In other embodiments, the toxin is attached to the NASM through a non-covalent bond.

In some embodiments, the NASM-toxin conjugate binds to target, thereby delivering toxin to the vicinity of the target. The toxin may interact with the same target, or with a second target in the vicinity of the first target.

In some embodiments, binding to the target results in the translocation of the NASM and associated toxin. For example, binding to the target results in the translocation of the NASM and associated toxin across a cell membrane. In some embodiments, binding to target results in the translocation of the NASM and associated toxin through structures in a organ, tissue or cell.

In some embodiments, the NASM-toxin conjugate binds to a target, and binding to target results in a change in conformation of the NASM-toxin conjugate. The change in conformation results in a change in activity of the NASM-toxin.

For example, in some embodiments, binding of the NASM-toxin conjugate to a target can result in a change in conformation of the NASM-toxin conjugate, such change resulting in a release in the toxin.

Alternatively, or in addition, binding of the NASM-toxin conjugate to a target can result in a change in conformation of the NASM-toxin conjugate, wherein the conformational change results in an activation of the toxin.

In a further embodiment, the NASM-toxin conjugate binds to a target, where binding to target results in a change in conformation of the NASM-toxin conjugate, and the change results in inactivation of the toxin.

In various embodiments, a NASM-toxin conjugate is provided whose half-life is less than, equal to, or greater than, the half-life of the toxin.

Also provided by the invention is a method of generating a NASM-toxin conjugate that includes attaching a toxin to a NASM. In some embodiments, the NASM in the moiety is created using a process similar to the SELEX process described above. However, rather than select for molecules with increased binding affinities, molecules are selected on the basis of their catalytic ability, i.e., their ability to turn the NASM on or off.

For example, the invention includes a method of generating a NASM-toxin conjugate by attaching a toxin to an a random pool of nucleic acids and then using the SELEX-like process described above to find the optimized NASM-toxin conjugate from within the random pool.

In some embodiments, the method of generating a NASM-toxin conjugate results in a NASM whose half-life is engineered to match the half life of the toxin. For example, the invention includes a method of generating a NASM-toxin conjugate where the NASM half life is engineered to match the half life of the toxin by adjusting the percentage of nuclease resistant bases in the NASM. In other embodiments, the invention includes a method of generating a NASM-toxin conjugate where the NASM half life is engineered to match the half life of the toxin by changing the 5′ and/or 3′ end capping.

The aptamer-toxins and/or NASM-toxins can be engineered so that the nucleic acid moiety recognizes a transporter, e.g., a folate transporter or an amino acid transporter (including a valine, arginine, lysine, or histidine transporter), a peptide transporter, a nucleotide transporter, or a sugar or carbohydrate transporter. Alternatively, or in addition, the nucleic acid moiety can be engineered to recognize a receptor that is internalized upon ligand binding, e.g., a receptor such as Her 2, EGF, glucose.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Nucleic Acid Compositions

In addition to carrying genetic information, nucleic acids can adopt complex three-dimensional structures. These three-dimensional structures are capable of specific recognition of target molecules and, furthermore, of catalyzing chemical reactions. Nucleic acids will thus provide candidate detection molecules for diverse target molecules, including those which do not naturally recognize or bind to DNA or RNA.

In aptamer selection, combinatorial libraries of oligonucleotides are screened in vitro to identify oligonucleotides which bind with high affinity to pre-selected targets. In NASM selection, on the other hand, combinational libraries of oligonucleotides are screened in vitro to identify oligonucleotides which exhibit increased catalytic activity in the presence of targets. Possible target molecules for both aptamers and NASMS include natural and synthetic polymers, including proteins, polysaccharides, glycoproteins, hormones, receptors, and cell surfaces, and small molecules such as drugs, metabolites, transition state analogs, specific phosphorylation states, and toxins. Small biomolecules, e.g., amino acids, nucleotides, NAD, S-adenosyl methionine, chloramphenicol, and large biomolecules, e.g., thrombin, Ku, DNA polymerases, are effective targets for aptamers, catalytic RNAs (ribozymes) discussed herein (e.g., hammerhead RNAs, hairpin RNAs) as well as NASMs.

While the aptamer selection processes described identifies aptamers through affinity-based (binding) selections, the selection processes as described for NASMs identifies nucleic acid sensor molecules through target modulation of the catalytic core of a ribozyme. In NASM selection, selective pressure on the starting population of NASMs (starting pool size is as high as 10¹⁴ to 10¹⁷ molecules) results in nucleic acid sensor molecules with enhanced catalytic properties, but not necessarily in enhanced binding properties. Specifically, the NASM selection procedures place selective pressure on catalytic effectiveness of potential NASMS by modulating both target concentration and reaction time-dependence. Either parameter, when optimized throughout the selection, can lead to nucleic acid molecular sensor molecules which have custom-designed catalytic properties, e.g., NASMs that have high switch factors, and or NASMs that have high specificity.

Aptamers

Systematic Evolution of Ligands by Exponential Enrichment, “SELEX™,” is a method for making a nucleic acid ligand for any desired target, as described, e.g., in U.S. Pat. Nos. 5,475,096; 5,670,637; 5,696,249; 5,270,163; 5,707,796; 5,595,877; 5,660,985; 5,567,588; 5,683,867; 5,637,459; 5,705,337; 6,011,020; 5,789,157; 6,261,774; EP 0 553 838 and PCT/US91/04078, each of which is specifically incorporated herein by reference.

SELEX™ technology is based on the fact that nucleic acids have sufficient capacity for forming a variety of two- and three-dimensional structures and sufficient chemical versatility available within their monomers to act as ligands (i.e., form specific binding pairs) with virtually any chemical compound, whether large or small in size.

The method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

Within a nucleic acid mixture containing a large number of possible sequences and structures, there is a wide range of binding affinities for a given target. A nucleic acid mixture comprising, for example a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEXTM until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20-50 nucleotides.

The basic SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes a SELEX™ based method for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. Each of these patents and applications is specifically incorporated herein by reference.

SELEX™ can also be used to obtain nucleic acid ligands that bind to more than one site on the target molecule, and to nucleic acid ligands that include non-nucleic acid species that bind to specific sites on the target.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

The random sequence portion of the oligonucleotide is flanked by at least one fixed sequence which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest.

In one embodiment, the random sequence portion of the oligonucleotide is about 15-70 (e.g., about 30-40) nucleotides in length and can comprise ribonucleotides and/or deoxyribonucleotides. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The SELEX™ method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described in U.S. Pat. No. 5,660,985, which describes oligonucleotides containing nucleotide derivatives chemically modified at the 5′ and 2′ positions of pyrimidines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described in U.S. Pat. No. 6,011,020.

SELEX™ identified nucleic acid ligands that are associated with a lipophilic compound, such as diacyl glycerol or dialkyl glycerol, in a diagnostic or therapeutic complex are described in U.S. Pat. No. 5,859,228. Nucleic acid ligands that are associated with a lipophilic compound, such as a glycerol lipid, or a non-immunogenic high molecular weight compound, such as polyalkylene glycol are further described in U.S. Pat. No. 6,051,698. See also PCT Publication No. WO 98/18480. These patents and applications allow the combination of a broad array of shapes and other properties, and the efficient amplification and replication properties, of oligonucleotides with the desirable properties of other molecules.

The identification of nucleic acid ligands to small, flexible peptides via the SELEXTM method has been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2-fluoro-ribonucleotide oligomer molecules can increase the sensitivity of an aptamer for a target molecule by ten- to- one hundred-fold over those generated using unsubstituted ribo- or deoxyribooligonucleotides (Pagratis, et al., Nat. Biotechnol. 15:68-73 (1997)), providing additional binding interactions with a target molecule and increasing the stability of the secondary structure(s) of the aptamer (Kraus, et al., Journal of Immunology 160:5209-5212 (1998); Pieken, et al., Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol 15:68-73 (1997)).

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase and purified. In one example, the 5′-fixed:random:3′-fixed sequence is separated by a random sequence having 30 to 50 nucleotides. Alternatively, the starting library can also be random RNA sequences synthesized on an RNA synthesizer.

Sorting among the billions of aptamer candidates to find the desired molecules starts from the complex sequence pool, whereby desired aptamers are isolated through an iterative in vitro selection process. The selection process removes both non-specific and non-binding types of contaminants. In a following amplification stage, thousands of copies of the surviving sequences are generated to enable the next round of selection. During amplification, random mutations can be introduced into the copied molecules—this ‘genetic noise’ allows functional nucleic acid aptamer molecules to continuously evolve and become even better adapted. The entire experiment reduces the pool complexity from 10¹⁷ molecules down to around 100 aptamer candidates that require detailed characterization.

Aptamer selection is accomplished by passing a solution of oligonucleotides through a column containing the target molecule. The flow-through, containing molecules which are incapable of binding target, is discarded. The column is washed, and the wash solution is discarded. Oligonucleotides which bound to the column are then specifically eluted, reverse transcribed, amplified by PCR (or other suitable amplification techniques), transcribed into RNA, and then reapplied to the selection column. Successive rounds of column application are performed until a pool of aptamers enriched in target binders is obtained.

Negative selection steps can also be performed during the selection process. Addition of such selection steps is useful to remove aptamers which bind to a target in addition to the desired target. Additionally, where the target column is known to contain an impurity, negative selection steps can be performed to remove from the binding pool those aptamers which bind selectively to the impurity, or to both the impurity and the desired target. For example, where the desired target is known, care must be taken so as to remove aptamers which bind to closely related molecules or analogs. Examples of negative selection steps include, for example, incorporating column washing steps with analogs in the buffer, or the addition of an analog column before the target selection column (e.g., the flow through from the analog column will contain aptamers which do not bind the analog).

After the completion of selection, the target-specific aptamers are reverse transcribed into DNA, cloned and amplified.

Aptamers can additionally include aptamer beacons as described, e.g., WO 00/70329. The publication discloses compositions, systems, and methods for simultaneously detecting the presence and quantity of one or more different compounds in a sample using aptamer beacons. Aptamer beacons are oligonucleotides that have a binding region that can bind to a non-nucleotide target molecule, such as a protein, a steroid, or an inorganic molecule. New aptamer beacons having binding regions configured to bind to different target molecules can be used in solution-based and solid, array-based systems. The aptamer beacons can be attached to solid supports, e.g., at different predetermined points in two-dimensional arrays.

2′Modified SELEX™

In addition, the SELEX™ method can be performed to generate 2′modified aptamers as described in U.S. Ser. No. 60/430,761, filed Dec. 3, 2002, U.S. Provisional Patent Application Ser. No. 60/487,474, filed Jul. 15, 2003, and U.S. Provisional Patent Application Ser. No. 60/517,039, filed Nov. 4, 2003, and U.S. patent application Ser. No. 10/729,581, filed Dec. 3, 2003, each of which is herein incorporated by reference in its entirety.

In order for an aptamer to be suitable for use as a therapeutic, it is preferably inexpensive to synthesize, safe and stable in vivo. Wild-type RNA and DNA aptamers are typically not stable in vivo because of their susceptibility to degradation by nucleases. Resistance to nuclease degradation can be greatly increased by the incorporation of modifying groups at the 2′-position. Fluoro and amino groups have been successfully incorporated into oligonucleotide libraries from which aptamers have been subsequently selected. However, these modifications greatly increase the cost of synthesis of the resultant aptamer, and may introduce safety concerns because of the possibility that the modified nucleotides could be recycled into host DNA, by degradation of the modified oligonucleotides and subsequent use of the nucleotides as substrates for DNA synthesis.

Aptamers that contain 2′-O-methyl (2′-OMe) nucleotides overcome many of these drawbacks. Oligonucleotides containing 2′-O-methyl nucleotides are nuclease-resistant and inexpensive to synthesize. Although 2′-O-methyl nucleotides are ubiquitous in biological systems, natural polymerases do not accept 2′-O-methyl NTPs as substrates under physiological conditions, thus there are no safety concerns over the recycling of 2′-O-methyl nucleotides into host DNA.

The present invention also provides materials and methods to produce stabilized oligonucleotides, including, e.g., aptamers, that contain modified nucleotides (e.g., nucleotides which have a modification at the 2′position) which make the oligonucleotide more stable than the unmodified oligonucleotide. The stabilized oligonucleotides produced by the materials and methods of the present invention are also more stable to enzymatic and chemical degradation as well as thermal and physical degradation. For example, oligonucleotides containing 2′-O-methyl nucleotides are nuclease-resistant and inexpensive to synthesize. Although 2′-O-methyl nucleotides are ubiquitous in biological systems, natural polymerases do not accept 2′-O-methyl NTPs as substrates under physiological conditions, thus there are no safety concerns over the recycling of 2′-O-methyl nucleotides into host DNA.

In one embodiment, the present invention provides combinations of 2′-OH, 2′-F, 2′-deoxy, and 2′-OMe modifications of the ATP, GTP, CTP, TTP, and UTP nucleotides. In another embodiment, the present invention provides combinations of 2′-OH, 2′-F, 2′-deoxy, 2′-OMe, 2′-NH₂, and 2′-methoxyethyl modifications of the ATP, GTP, CTP, TTP, and UTP nucleotides. In one embodiment, the present invention provides 56 combinations of 2′-OH, 2′-F, 2′-deoxy, 2′-OMe, 2′-NH₂, and 2′-methoxyethyl modifications the ATP, GTP, CTP, TTP, and UTP nucleotides.

2′ modified aptamers of the invention are created using modified polymerases, such as, e.g., a modified T7 polymerase, having a higher incorporation rate of modified nucleotides having bulky substituents at the furanose 2′ position, than wild-type polymerases. For example, a double T7 polymerase mutant (Y639F/H784A) having the histidine at position 784 changed to an alanine, or other small amino acid, residue, in addition to the Y639F mutation has been described for incorporation of bulky 2′ substituents and has been used to incorporate modified pyrimidine NTPs. A single mutant T7 polymerase (H784A) having the histidine at position 784 changed to an alanine residue has also been described. (Padilla et al., Nucleic Acids Research, 2002, 30:138). In both the Y639F/H784A double mutant and H784A single mutant T7 polymerases, the change to smaller amino acid residues allows for the incorporation of bulkier nucleotide substrates, e.g., 2′-O methyl substituted nucleotides.

Another important factor in the production of 2′-modified aptamers is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

There are several examples of 2′-OMe containing aptamers in the literature, see, for example Green et al., Current Biology 2, 683-695, 1995. These were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution, and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, aptamers generated using this method tend to contain from two to four 2′-OH residues, and stability and cost of synthesis are compromised as a result. By incorporating modified nucleotides into the transcription reaction which generate stabilized oligonucleotides used in oligonucleotide libraries from which aptamers are selected and enriched by SELEX™ (and/or any of its variations and improvements, including those described below), the methods of the present invention eliminate the need for stabilizing the selected aptamer oligonucleotides (e.g., by resynthesizing the aptamer oligonucleotides with modified nucleotides).

Furthermore, the modified oligonucleotides of the invention can be further stabilized after the selection process has been completed. (See “post-SELEX™ modifications”, including truncating, deleting and modification, below.)

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

Incorporation of modified nucleotides into the aptamers of the invention is accomplished before (pre-) the selection process (e.g., a pre-SELEX™ process modification). Optionally, aptamers of the invention in which modified nucleotides have been incorporated by pre-SELEX™ process modification can be further modified by post-SELEX™ process modification (i.e., a post-SELEX™ process modification after a pre-SELEX™ modification). Pre-SELEX™ process modifications yield modified nucleic acid ligands with specificity for the SELEX™ target and also improved in vivo stability. Post-SELEX™ process modifications (e.g., modification of previously identified ligands having nucleotides incorporated by pre-SELEX™ process modification) can result in a further improvement of in vivo stability without adversely affecting the binding capacity of the nucleic acid ligand having nucleotides incorporated by pre-SELEX™ process modification.

Modified Polymerases

A single mutant T7 polymerase (Y639F) in which the tyrosine residue at position 639 has been changed to phenylalanine readily utilizes 2′deoxy, 2′amino-, and 2′fluoro-nucleotide triphosphates (NTPs) as substrates and has been widely used to synthesize modified RNAs for a variety of applications. However, this mutant T7 polymerase reportedly can not readily utilize (e.g., incorporate) NTPs with bulkier 2′-substituents, such as 2′-O-methyl (2′-OMe) or 2′-azido (2′-N₃) substituents. For incorporation of bulky 2′ substituents, a double T7 polymerase mutant (Y639F/H784A) having the histidine at position 784 changed to an alanine, or other small amino acid, residue, in addition to the Y639F mutation has been described and has been used to incorporate modified pyrimidine NTPs. A single mutant T7 polymerase (H784A) having the histidine at position 784 changed to an alanine residue has also been described. (Padilla et al., Nucleic Acids Research, 2002, 30: 138). In both the Y639F/H784A double mutant and H784A single mutant T7 polymerases, the change to smaller amino acid residues allows for the incorporation of bulkier nucleotide substrates, e.g., 2′-O methyl substituted nucleotides.

The present invention provides methods and conditions for using these and other modified T7 polymerases having a higher incorporation rate of modified nucleotides having bulky substituents at the furanose 2′ position, than wild-type polymerases. Generally, it has been found that under the conditions disclosed herein, the Y693F single mutant can be used for the incorporation of all 2′-OMe substituted NTPs except GTP and the Y639F/H784A double mutant can be used for the incorporation of all 2′-OMe substituted NTPs including GTP. It is expected that the H784A single mutant possesses similar properties when used under the conditions disclosed herein.

The present invention provides methods and conditions for modified T7 polymerases to enzymatically incorporate modified nucleotides into oligonucleotides. Such oligonucleotides may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or libraries of transcripts are generated using any combination of modifications, for example, ribonucleotides, (2′-OH, “rN”), deoxyribonucleotides (2′-deoxy), 2′-F, and 2′-OMe nucleotides. A mixture containing 2′-OMe C and U and 2′-OH A and G is called “rRmY”; a mixture containing deoxy A and G and 2′-OMe U and C is called “dRmY”; a mixture containing 2′-OMe A, C, and U, and 2′-OH G is called “rGmH”; a mixture alternately containing 2′-OMe A, C, U and G and 2′-OMe A, U and C and 2′-F G is called “toggle”; a mixture containing 2′-OMe A, U, C, and G, where up to 10% of the G's are deoxy is called “r/mGmH”; a mixture containing 2′-O Me A, U, and C, and 2′-F G is called “fGmH”; and a mixture containing deoxy A, and 2′-OMe C, G and U is called “dAmB”.

A preferred embodiment includes any combination of 2′-OH, 2′-deoxy and 2′-OMe nucleotides. A more preferred embodiment includes any combination of 2′-deoxy and 2′-OMe nucleotides. An even more preferred embodiment is with any combination of 2′-deoxy and 2′-OMe nucleotides in which the pyrimidines are 2′-OMe (such as dRmY, mN or dGmH).

2′-O-Methyl Modified Nucleotide SELEX™

The present invention provides methods to generate libraries of 2′-modified (e.g., 2′-OMe) RNA transcripts in conditions under which a polymerase accepts 2′-modified NTPs. Preferably, the polymerase is the Y693F/H784A double mutant or the Y693F single mutant. Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein. A number of factors have been determined to be crucial for the transcription conditions useful in the methods disclosed herein. For example, great increases in the yields of modified transcript are observed when a leader sequence is incorporated into the 5′ end of a fixed sequence at the 5′ end of the DNA transcription template, such that at least about the first 6 residues of the resultant transcript are all purines.

Another important factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides, the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of principally the 2′-OMe GTP.

Another important factor in the incorporation of 2′-OMe into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of maximally 2′ substituted O-methylated transcripts (i.e., all A, C, and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

For maximum incorporation of 2′-OMe ATP (100%), UTP(100%), CTP(100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (6.5 mM where the concentration of each 2′-OMe NTP is 1.0 mM), MnCl₂ 1.5 mM (2.0 mM where the concentration of each 2′-OMe NTP is 1.0 mM), 2′-OMe NTP (each) 500 μM (more preferably, 1.0 mM), 2′-OH GTP 30 pM, 2′-OH GMP 500 μM, pH 7.5, Y639F/H784A T7 RNA Polymerase 15 units/ml, inorganic pyrophosphatase 5 units/ml, and an all-purine leader sequence of at least 8 nucleotides long. As used herein, one unit of the Y639F/H784A mutant T7 RNA polymerase, or any other mutant T7 RNA polymerase specified herein) is defined as the amount of enzyme required to incorporate 1 mmole of 2′-OMe NTPs into transcripts under the r/mGmH conditions. As used herein, one unit of inorganic pyrophosphatase is defined as the amount of enzyme that will liberate 1.0 mole of inorganic orthophosphate per minute at pH 7.2 and 25° C.

For maximum incorporation (100%) of 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F/H784A T7 RNA Polymerase 15 units/ml, inorganic pyrophosphatase 5 units/ml, and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long. Optionally, 2 mM spermine is added.

For each of the above, (1) transcription is preferably performed at a temperature of from about 30° C. to about 45° C. and for a period of at least two hours and (2) 50-300 nM of a double stranded DNA transcription template is used (200 nm template was used for round 1 to increase diversity (300 nm template was used for dRmY transcriptions), and for subsequent rounds approximately 50 nM, a 1/10 dilution of an optimized PCR reaction, using conditions described herein, was used). The preferred DNA transcription templates are described below (where ARC254 and ARC256 transcribe under all 2′-OMe conditions and ARC255 transcribes under rRmY conditions).

| | |
|---|---|
| ARC254: | |
| 5′-CATCGATGCTAGTCGTAACGATCCNNNNNNNNN | (SEQ ID NO: 1) |
| NNNNNNNNNNNNNNNNNNNNNCGAGAACGTTCTCTC | |
| CTCTCCCTATAGTGAGTCGTATTA-3′ | |
| ARC255: | |
| 5′-CATGCATCGCGACTGACTAGCCGNNNNNNNNNN | (SEQ ID NO: 2) |
| NNNNNNNNNNNNNNNNNNNNGTAGAACGTTCTCTCC | |
| TCTCCCTATAGTGAGTCGTATTA-3′ | |
| ARC256: | |
| 5′-CATCGATCGATCGATCGACAGCGNNNNNNNNNN | (SEQ ID NO: 3) |
| NNNNNNNNNNNNNNNNNNNNGTAGAACGTTCTCTCC | |
| TCTCCCTATAGTGAGTCGTATTA-3′ | |

Under rN transcription conditions of the present invention, the transcription reaction mixture comprises 2′-OH adenosine triphosphates (ATP), 2′-OH guanosine triphosphates (GTP), 2′-OH cytidine triphosphates (CTP), and 2′-OH uridine triphosphates (UTP). The modified oligonucleotides produced using the rN transcription mixtures of the present invention comprise substantially all 2′-OH adenosine, 2′-OH guanosine, 2′-OH cytidine, and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine, and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine, and at least 90% of all uridine nucleotides are 2′-OH uridine. In a most preferred embodiment of rN transcription, the modified oligonucleotides of the present invention comprise 100% of all adenosine nucleotides are 2′-OH adenosine, of all guanosine nucleotides are 2′-OH guanosine, of all cytidine nucleotides are 2′-OH cytidine, and of all uridine nucleotides are 2′-OH uridine.

Under rRmY transcription conditions of the present invention, the transcription reaction mixture comprises 2′-OH adenosine triphosphates, 2′-OH guanosine triphosphates, 2′-O-methyl cytidine triphosphates, and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In a preferred embodiment, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions of the present invention, the transcription reaction mixture comprises 2′-deoxy purine triphosphates and 2′-O-methylpyrimidine triphosphates. The modified oligonucleotides produced using the dRmY transcription conditions of the present invention comprise substantially all 2′-deoxy purines and 2′-O-methylpyrimidines. In a preferred embodiment, the resulting modified oligonucleotides of the present invention comprise a sequence where at least 80% of all purine nucleotides are 2′-deoxy purines and at least 80% of all pyrimidine nucleotides are 2′-O-methylpyrimidines. In a more preferred embodiment, the resulting modified oligonucleotides of the present invention comprise a sequence where at least 90% of all purine nucleotides are 2′-deoxy purines and at least 90% of all pyrimidine nucleotides are 2′-O-methylpyrimidines. In a most preferred embodiment, the resulting modified oligonucleotides of the present invention comprise a sequence where 100% of all purine nucleotides are 2′-deoxy purines and 100% of all pyrimidine nucleotides are 2′-O-methyl pyrimidines.

Under rGmH transcription conditions of the present invention, the transcription reaction mixture comprises 2′-OH guanosine triphosphates, 2′-O-methyl cytidine triphosphates, 2′-O-methyl uridine triphosphates, and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine, and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

Under r/mGmH transcription conditions of the present invention, the transcription reaction mixture comprises 2′-O-methyl adenosine triphosphate, 2′-O-methyl cytidine triphosphate, 2′-O-methyl guanosine triphosphate, 2′-O-methyl uridine triphosphate and deoxy guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine, and 2′-O-methyl uridine, wherein the population of guanosine nucleotides has a maximum of about 10% deoxy guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine, and no more than about 10% of all guanosine nucleotides are deoxy guanosine.

Under fGmH transcription conditions of the present invention, the transcription reaction mixture comprises 2′-O-methyl adenosine triphosphates (ATP), 2′-O-methyl uridine triphosphates (UTP), 2′-O-methyl cytidine triphosphates (CTP), and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine, and 2′-F guanosine. In a preferred embodiment, the resulting modified oligonucleotides comprise a sequence where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. The resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

Under dAmB transcription conditions of the present invention, the transcription reaction mixture comprises 2′-deoxy adenosine triphosphates (dATP), 2′-O-methyl cytidine triphosphates (CTP), 2′-O-methyl guanosine triphosphates (GTP), and 2′-O-methyl uridine triphosphates (UTP). The modified oligonucleotides produced using the dAmB transcription mixtures of the present invention comprise substantially all 2′-deoxy adenosine, 2′-O-methyl cytidine, 2′-O-methyl guanosine, and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

In each case, the transcription products can then be used as the library in the SELEX™ process to identify aptamers and/or to determine a conserved motif of sequences that have binding specificity to a given target. The resulting sequences are already stabilized, eliminating this step from the process to arrive at a stabilized aptamer sequence and giving a more highly stabilized aptamer as a result. Another advantage of the 2′-OMe SELEX™ process is that the resulting sequences are likely to have fewer 2′-OH nucleotides required in the sequence, possibly none.

As described below, lower but still useful yields of transcripts fully incorporating 2′-OMe substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10, for example without limitation, Tris(hydroxymethyl)aminomethane.

The DTT concentration can range from 0 to 400 mM. The methods of the present invention also provide for the use of other reducing agents, for example without limitation, mercaptoethanol.

The spermidine and/or spermine concentration can range from 0 to 20 mM.

The PEG-8000 concentration can range from 0 to 50% (w/v). The methods of the present invention also provide for the use of other hydrophilic polymer, for example without limitation, other molecular weight PEG or other polyalkylene glycols.

The Triton X-100 concentration can range from 0 to 0.1% (w/v). The methods of the present invention also provide for the use of other non-ionic detergents, for example without limitation, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5, more preferably, the ratio is about 3 to about 4.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides. In addition, the methods of the present invention provide for the optional use of chelating agents in the transcription reaction condition, for example without limitation, EDTA, EGTA, and DTT.

The selected aptamers having the highest affinity and specific binding as demonstrated by biological assays as described in the examples below are suitable therapeutics for treating conditions in which the target is involved in pathogenesis.

Aptamer Therapeutics

Aptamers represent a promising class of therapeutic agents currently in pre-clinical and clinical development. Like biologics, e.g., peptides or monoclonal antibodies, aptamers are capable of binding specifically to molecular targets and, through binding, inhibiting target function. A typical aptamer is 10-15 kDa in size (i.e., 30-45 nucleotides), binds its target with sub-nanomolar affinity, and discriminates among closely related targets (e.g., will typically not bind other proteins from the same gene family) (Griffin, et al. (1993), Gene 137(1): 25-31; Jenison, et al. (1998), Antisense Nucleic Acid Drug Dev. 8(4): 265-79; Bell, et al. (1999), In Vitro Cell. Dev. Biol. Anim. 35(9): 533-42; Watson, et al. (2000), Antisense Nucleic Acid Drug Dev. 10(2): 63-75; Daniels, et al. (2002), Anal. Biochem. 305(2): 214-26; Chen, et al. (2003), Proc. Natl. Acad. Sci. U.S.A. 100(16): 9226-31; Khati, et al. (2003), J. Virol. 77(23): 12692-8; Vaish, et al. (2003), Biochemistry 42(29): 8842-51). Created by an entirely in vitro selection process (SELEX) from libraries of random sequence oligonucleotides, aptamers have been generated against numerous proteins of therapeutic interest, including growth factors, enzymes, immunoglobulins, and receptors (Ellington and Szostak (1990), Nature 346(6287): 818-22; Tuerk and Gold (1990), Science 249(4968): 505-510).

Aptamers have a number of attractive characteristics for use as therapeutics. In addition to high target affinity and specificity, aptamers have shown little or no toxicity or immunogenicity in standard assays (Wlotzka, et al. (2002), Proc. Natl. Acad. Sci. U.S.A. 99(13): 8898-902). Several therapeutic aptamers have been optimized and advanced through varying stages of pre-clinical development, including pharmacokinetic analysis, characterization of biological efficacy in cellular and animal disease models, and preliminary safety pharmacology assessment (Reyderman and Stavchansky (1998), Pharmaceutical Research 15(6): 904-10; Tucker et al., (1999), J. Chromatography B. 732: 203-212; Watson, et al. (2000), Antisense Nucleic Acid Drug Dev. 10(2): 63-75).

It is important that the pharmacokinetic properties for all oligonucleotide-based therapeutics, including aptamers, be tailored to match the desired pharmaceutical application. While aptamers directed against extracellular targets do not suffer from difficulties associated with intracellular delivery (as is the case with antisense and RNAi-based therapeutics), such aptamers must be able to be distributed to target organs and tissues, and remain in the body (unmodified) for a period of time consistent with the desired dosing regimen. Early work on nucleic acid-based therapeutics has shown that, while unmodified oligonucleotides are degraded rapidly by nuclease digestion, protective modifications at the 2′-position of the sugar, and use of inverted terminal cap structures, e.g., [3′-3′dT], dramatically improve nucleic acid stability in vitro and in vivo (Green, et al. (1995), Chem. Biol. 2(10): 683-95; Jellinek, et al. (1995), Biochemistry 34(36): 11363-72; Ruckman, et al. (1998), J. Biol. Chem. 273(32): 20556-67; Uhlmann, et al. (2000), Methods Enzymol. 313: 268-84). In some SELEX selections (i.e., SELEX experiments or SELEXions), starting pools of nucleic acids from which aptamers are selected are typically pre-stabilized by chemical modification, for example by incorporation of 2′-fluoropyrimidine (2′-F) substituted nucleotides, to enhance resistance of aptamers against nuclease attack. Aptamers incorporating 2′-O-methylpurine (2′-O-Me purine) substituted nucleotides have also been developed through post-SELEX modification steps or, more recently, by enabling synthesis of 2′-O-Me-containing random sequence libraries as an integral component of the SELEX process itself, as described above.

In addition to clearance by nucleases, oligonucleotide therapeutics are subject to elimination via renal filtration. As such, a nuclease-resistant oligonucleotide administered intravenously exhibits an in vivo half-life of <10 min, unless filtration can be blocked. This can be accomplished by either facilitating rapid distribution out of the blood stream into tissues or by increasing the apparent molecular weight of the oligonucleotide above the effective size cut-off for the glomerulus. Conjugation of small therapeutics to a PEG polymer (PEGylation), described below, can dramatically lengthen residence times of aptamers in circulation, thereby decreasing dosing frequency and enhancing effectiveness against vascular targets. Previous work in animals has examined the plasma pharmacokinetic properties of PEG-conjugated aptamers (Reyderman and Stavchansky (1998), Pharmaceutical Research 15(6): 904-10; Watson, et al. (2000), Antisense Nucleic Acid Drug Dev. 10(2): 63-75). Determining the extravasation of an aptamer therapeutic, including aptamer therapeutics conjugated to a modifying moiety or containing modified nucleotides, and in particular, determining the potential of aptamers or their modified forms to access diseased tissues (for example, sites of inflammation, or the interior of tumors) will better define the spectrum of therapeutic opportunities for aptamer intervention.

The pharmacokinetic profiles of aptamer compositions of the invention have “tunability” (i.e., the ability to modulate aptamer pharmacokinetics). The tunability of aptamer pharmacokinetics is achieved, for example through conjugation of modifying moieties (e.g., PEG polymers) to the aptamer and/or the incorporation of modified nucleotides (e.g., 2′-fluoro and/or 2′-O-Me substitutions) to alter the chemical composition of the nucleic acid.

In addition, the tunability of aptamer pharmacokinetics is used to modify the biodistribution of an aptamer therapeutic in a subject. For example, in some therapeutic applications, it may be desirable to alter the biodistribution of an aptamer therapeutic in an effort to target a particular type of tissue or a specific organ (or set of organs). In these applications, the aptamer therapeutic preferentially accumulates in a specific tissue or organ(s). In other therapeutic applications, it may be desirable to target tissues displaying a cellular marker or a symptom associated with a given disease, cellular injury or other abnormal pathology, such that the aptamer therapeutic preferentially accumulates in the affected tissue. For example, as described in copending provisional application U.S. Ser. No. 60/550,790, filed on Mar. 5, 2004 and entitled “Controlled Modulation of the Pharmacokinetics and Biodistribution of Aptamer Therapeutics”, PEGylation of an aptamer therapeutic (e.g. PEGylation with a 20 kDa PEG polymer) is used to target inflamed tissues, such that the PEGylated aptamer therapeutic preferentially accumulates in inflamed tissue.

The pharmacokinetic and biodistribution profiles of aptamer therapeutics are determined by monitoring a variety of parameters. Such parameters include, for example, the half-life (t_{1/2}), the plasma clearance (C1), the volume of distribution (Vss), the area under the concentration-time curve (AUC), maximum observed serum or plasma concentration (Cₘₐₓ), and the mean residence time (MRT) of an aptamer composition. As used herein, the term “AUC” refers to the area under the plot of the plasma concentration of an aptamer therapeutic versus the time after aptamer administration. The AUC value is used to estimate the bioavailability (i.e., the percentage of administered aptamer therapeutic in the circulation after aptamer administration) and/or total clearance (C1) (i.e., the rate at which the aptamer therapeutic is removed from circulation) of a given aptamer therapeutic. The volume of distribution relates the plasma concentration of an aptamer therapeutic to the amount of aptamer present in the body. The larger the Vss, the more an aptamer is found outside of the plasma (i.e., the more extravasation).

Modulation of Pharmacokinetics and Biodistribution of Aptamer Therapeutics

The present invention provides materials and methods to affect the pharmacokinetics of aptamer compositions, and, in particular, the ability to tune (i.e., the “tunability”) aptamer pharmacokinetics. The tunability of aptamer pharmacokinetics is achieved through conjugation of modifying moieties to the aptamer and/or the incorporation of modified nucleotides to alter the chemical composition of the nucleic acid. The ability to tune aptamer pharmacokinetics is used in the improvement of existing therapeutic applications, or alternatively, in the development of new therapeutic applications. For example, in some therapeutic applications, e.g., in anti-neoplastic or acute care settings where rapid drug clearance or turn-off may be desired, it is desirable to decrease the residence times of aptamers in the circulation. Alternatively, in other therapeutic applications, e.g., maintenance therapies where systemic circulation of a therapeutic is desired, it may be desirable to increase the residence times of aptamers in circulation.

In addition, the tunability of aptamer pharmacokinetics is used to modify the biodistribution of an aptamer therapeutic in a subject. For example, in some therapeutic applications, it may be desirable to alter the biodistribution of an aptamer therapeutic in an effort to target a particular type of tissue or a specific organ (or set of organs). In these applications, the aptamer therapeutic preferentially accumulates in a specific tissue or organ(s). In other therapeutic applications, it may be desirable to target tissues displaying a cellular marker or a symptom associated with a given disease, cellular injury or other abnormal pathology, such that the aptamer therapeutic preferentially accumulates in the affected tissue. For example, as described herein, PEGylation of an aptamer therapeutic (e.g. PEGylation with a 20 kDa PEG polymer) is used to target inflamed tissues, such that the PEGylated aptamer therapeutic preferentially accumulates in inflamed tissue.

The pharmacokinetic and biodistribution profiles of aptamer therapeutics (e.g., aptamer conjugates or aptamers having altered chemistries, such as modified nucleotides) are determined by monitoring a variety of parameters. Such parameters include, for example, the half-life (t_{1/2}), the plasma clearance (C1), the volume of distribution (Vss), the area under the concentration-time curve (AUC), maximum observed serum or plasma concentration (Cₘₐₓ), and the mean residence time (MRT) of an aptamer composition. As used herein, the term “AUC” refers to the area under the plot of the plasma concentration of an aptamer therapeutic versus the time after aptamer administration. The AUC value is used to estimate the bioavailability (i.e., the percentage of administered aptamer therapeutic in the circulation after aptamer administration) and/or total clearance (C1) (i.e., the rate at which the aptamer therapeutic is removed from circulation) of a given aptamer therapeutic. The volume of distribution relates the plasma concentration of an aptamer therapeutic to the amount of aptamer present in the body. The larger the Vss, the more an aptamer is found outside of the plasma (i.e., the more extravasation).

The pharmacokinetic and biodistribution properties of phosphorothioate-containing antisense oligonucleotides, which clear rapidly from circulation, and distribute into tissues (where elimination occurs slowly, as a result of metabolic degradation) are described in the art: (See e.g., Srinivasan and Iversen (1995), J. Clin. Lab. Anal. 9(2): 129-37; Agrawal and Zhang (1997), Ciba Found. Symp. 209: 60-75, discussion 75-8; Akhtar and Agrawal (1997), Trends Pharmacol. Sci. 18(1): 12-8; Crooke (1997), Adv. Pharmacol. 40: 1-49; Grindel, et al. (1998), Antisense Nucleic Acid Drug Dev. 8(1): 43-52; Monteith and Levin (1999), Toxicol. Pathol. 27(1): 8-13; Peng, et al. (2001), Antisense Nucleic Acid Drug Dev. 11(1): 15-27). Early studies involving antisense oligonucleotides have explored the effects of various conjugation chemistries on pharmacokinetics and biodistribution, with the ultimate goal of increasing delivery of antisense molecules to their sites of action inside cells or within certain tissue types in vivo (Antopolsky, et al. (1999), Bioconjug. Chem. 10(4): 598-606; Zubin, et al. (1999), FEBS Lett. 456(1): 59-62; Astriab-Fisher, et al. (2000), Biochem. Pharmacol. 60(1): 83-90; Lebedeva, et al. (2000), Eur. J. Pharm. Biopharm. 50(1): 101-19; Manoharan (2002), Antisense Nucleic Acid Drug Dev. 12(2): 103-28). For example, conjugation with cholesterol has been reported to increase the circulation half-life of antisense oligonucleotides, most likely through association with plasma lipoproteins, and promoting hepatic uptake (de Smidt, et al. (1991), Nucleic Acids Res. 19(17): 4695-4700). Early work involving antisense oligonucleotides has indicated that nonspecific protein-binding interactions play an important role in the rapid loss of phosphorothioate-containing antisense oligonucleotide from circulation and distribution to tissues (See e.g., Srinivasan and Iversen (1995), J. Clin. Lab. Anal. 9(2): 129-37; Agrawal and Zhang (1997), Ciba Found. Symp. 209: 60-75, discussion 75-8; Akhtar and Agrawal (1997), Trends Pharmacol. Sci 18(1): 12-8; Crooke (1997), Adv. Pharmacol. 40: 1-49; Grindel, et al. (1998), Antisense Nucleic Acid Drug Dev. 8(1): 43-52; Monteith and Levin (1999), Toxicol. Pathol. 27(1): 8-13; Peng, et al. (2001), Antisense Nucleic Acid Drug Dev. 11 (1): 15-27).

In contrast to antisense oligonucleotides, aptamers are generally longer (30-40 vs. 10-20 nucleotides), possess different types of chemical modifications (sugar modifications, e.g., 2′-F, 2′-O-Me, 2′-NH₂, vs. backbone modifications), and assume complex tertiary structures that are more resistant to degradation. Aptamers are, in many respects, more structurally similar to the three dimensional forms of globular proteins than to nucleic acids. Given these considerable differences, the in vivo disposition of aptamers is not readily predictable from antisense results.

More recently, delivery peptides for carrying large polar macromolecules, including oligonucleotides, across cellular membranes have also been explored as a means to augment in vivo the range for application of antisense and other therapeutics. Examples of these conjugates include a 13-amino acid fragment (Tat) of the HIV Tat protein (Vives, et al. (1997), J. Biol. Chem. 272(25): 16010-7), a 16-amino acid sequence derived from the third helix of the Drosophila antennapedia (Ant) homeotic protein (Pietersz, et al. (2001), Vaccine 19(11-12): 1397-405), and short, positively charged cell-permeating peptides composed of polyarginine (Arg7) (Rothbard, et al. (2000), Nat. Med. 6(11): 1253-7; Rothbard, J et al. (2002), J. Med. Chem. 45(17): 3612-8). For example, the TAT peptide is described in U.S. Pat. Nos. 5,804,604 and 5,674,980.

The present invention provides materials and methods to modulate, in a controlled manner, the pharmacokinetics and biodistribution of stabilized aptamer compositions in vivo by conjugating an aptamer to a modulating moiety such as a small molecule, peptide, or polymer terminal group, or by incorporating modified nucleotides into an aptamer. Pharmacokinetics and biodistribution of aptamer conjugates in biological samples are quantified radiometrically and by a hybridization-based dual probe capture assay with enzyme-linked fluorescent readout. As described herein, conjugation of a modifying moiety and/or altering nucleotide(s) chemical composition alter fundamental aspects of aptamer residence time in circulation and distribution to tissues.

Aptamers are conjugated to a variety of modifying moieties, such as, for example, high molecular weight polymers, e.g., PEG, peptides, e.g., Tat, Ant and Arg₇, and small molecules, e.g., lipophilic compounds such as cholesterol. As shown herein, a mixed composition aptamer containing both 2° F. and 2′-O-Me stabilizing modifications persisted significantly longer in the blood stream than did a fully 2′-O-methylated composition. Among the conjugates prepared according to the materials and methods of the present invention, in vivo properties of aptamers were altered most profoundly by complexation with PEG groups. For example complexation of the mixed 2° F. and 2′-O-Me modified aptamer therapeutic with a 20 kDa PEG polymer hindered renal filtration and promoted aptamer distribution to both healthy and inflamed tissues. Furthermore, the 20 kDa PEG polymer-aptamer conjugate proved nearly as effective as a 40 kDa PEG polymer in preventing renal filtration of aptamers. While one effect of PEGylation was on aptamer clearance, the prolonged systemic exposure afforded by presence of the 20 kDa moiety also facilitated distribution of aptamer to tissues, particularly those of highly perfused organs and those at the site of inflammation. The aptamer-20 kDa PEG polymer conjugate (ARC120) directed aptamer distribution to the site of inflammation, such that the PEGylated aptamer preferentially accumulated in inflamed tissue. In some instances, the 20 kDa PEGylated aptamer conjugate was able to access the interior of cells, such as, for example, kidney cells.

Overall, effects on aptamer pharmacokinetics and tissue distribution produced by low molecular weight modifying moieties, including cholesterol and cell-permeating peptides were less pronounced than those produced as a result of PEGylation or modification of nucleotides (e.g., an altered chemical composition). An aptamer conjugated to cholesterol showed more rapid plasma clearance relative to unconjugated aptamer, and a large volume of distribution, which suggests some degree of aptamer extravasation. This result appears to contrast published data demonstrating the capacity of a cholesterol tag to significantly prolong the plasma half-life of an antisense oligonucleotide (de Smidt et al., (1991), Nucleic Acids Res. 19(17): 4695-4700). While not intending to be bound by theory, the results provided herein, may suggest that cholesterol-mediated associations with plasma lipoproteins, postulated to occur in the case of the antisense conjugate, are precluded in the particular context of the aptamer-cholesterol conjugate folded structure, and/or relate to aspect of the lipophilic nature of the cholesterol group. Like cholesterol, the presence of a Tat peptide tag promoted clearance of aptamer from the blood stream, with comparatively high levels of conjugate appearing in the kidneys at 48 hrs. Other peptides (e.g., Ant, Arg₇) that have been reported in the art to mediate passage of macromolecules across cellular membranes in vitro, did not appear to promote aptamer clearance from circulation. However, like Tat, the Ant conjugate significantly accumulated in the kidneys relative to other aptamers. While not intending to be bound by theory, it is possible that unfavorable presentation of the Ant and Arg₇ peptide modifying moieties in the context of three dimensionally folded aptamers in vivo impaired the ability of these peptides to influence aptamer transport properties.

Prior to the invention described herein, little was known concerning the pharmacokinetics and biodistribution of oligonucleotides with a 2′-O-Me chemical composition (Tavitian, et al. (1998), Nat. Med. 4(4): 467-71). For several reasons, incorporation of 2′-O-Me substitutions is a particularly attractive means to stabilize aptamers against nuclease attack. One attribute is safety: 2′-O-methylation is known as a naturally occurring and abundant chemical modification in eukaryotic ribosomal and cellular RNAs. Human rRNAs are estimated to contain roughly one hundred 2′-O-methylated sugars per ribosome (Smith and Steitz (1997), Cell 89(5): 669-72). Thus, aptamer compositions incorporating 2′-O-Me substitutions are expected to be non-toxic. In support of this view, in vitro and in vivo studies indicate that 2′-O-Me nucleotides are not readily polymerized by human DNA polymerases (α or γ), or by human DNA primase, and thus, pose a low risk for incorporation into genomic DNA (Richardson, et al. (2000), Biochem. Pharmacol. 59(9): 1045-52; Richardson, et al. (2002), Chem. Res. Toxicol. 15(7): 922-6). Additionally, from a cost of goods perspective, pricing per gram for synthesis of 2′-O-Me containing oligonucleotides is less than the pricing per gram for both 2′-F and 2′-OH containing RNAs.

A comparison of a mixed 2° F./2′-O-Me composition aptamer and conjugated aptamers was conducted in vivo to determine plasma clearance. The unconjugated test aptamer which incorporates both 2′-F and 2′-O-Me stabilizing chemistries, is typical of current generation aptamers as it exhibits a high degree of nuclease stability in vitro and in vivo. Compared to the mixed 2° F./2′-O-Me composition aptamer, unmodified aptamer displayed rapid loss from plasma (i.e., rapid plasma clearance) and a rapid distribution into tissues, primarily into the kidney.

Tests can be conducted to determine whether the hydrophobic nature of a fully 2′-O-Me modified aptamer renders the oligonucleotide more prone to nonspecific associations with plasma or cellular component (as is the case with antisense oligonucleotides). In addition, experiments can be conducted to define the protein-binding properties of 2′-O-Me-modified aptamers. While not intending to be bound by theory, levels of full-length all-2′-O-methyl substituted aptamer above background were detected in several tissues, kidney, liver, and spleen, even at 48 hrs after dosing, possibly due to the extreme robustness of the fully 2′-O-Me aptamer towards nuclease digestion. Consistent with its plasma clearance profile and distribution to the kidney, the fully 2′-O-Me aptamer, ARC 159, was eliminated rapidly via the urine.

When expressed as percent of administered dose, all aptamers or conjugates examined herein showed significant levels of distribution to kidney, liver, and gastrointestinal tract. When corrected for organ/tissue weight, highest mass-normalized concentrations of aptamers were seen in highly perfused organs (kidneys, liver, spleen, heart, lungs) and unexpectedly, mediastinal lymph nodes. Since aptamers are bioavailable (up to 80%) following subcutaneous injection (Tucker et al., (1999), J. Chromatography B. 732: 203-212), they are expected to have access to targets in the lymphatic system through this route of administration. Ready access to the lymphatics via intravenous dosing is of interest from the standpoint of developing aptamer therapeutics for infectious disease indications such as HIV/AIDS. Thus, aptamer therapeutics conjugated to modifying moieties and aptamers having altered chemistries (e.g., including modified nucleotides) will be useful in the treatment of infectious diseases such as HIV/AIDS.

Consistent with its enhanced plasma pharmacokinetics, the concentration of 20 kDa PEGylated aptamer detected in highly perfused organs was higher than for the other aptamers that were assayed. As a general trend, aptamer concentrations measured in the kidneys decreased with time, with exception of 20 kDa PEGylated aptamer, where concentrations remained roughly constant over time. Conversely, in liver concentrations of all aptamers remained roughly constant, except for 20 kDa PEGylated aptamer, whose levels decreased with time. These differences may be understood in terms of the extended plasma half-life of the 20 kDa PEG conjugate and its increased uptake in highly perfused organs. While one of the effects of complexation with a 20 kDa PEG modifying moiety was to retard renal filtration of the aptamer conjugate, the comparatively high concentrations of the 20 kDa PEG conjugate measured in well-perfused organs, relative to other aptamers or conjugates, suggested that PEGylation can modulate aptamer distribution to tissues, as well as promote extended plasma half-life (t_{1/2}). As described herein, the 20 kDa PEGylated aptamer-conjugate modulated aptamer distribution to tissues. The level of the 20 kDa PEGylated aptamer detected in inflamed tissues was higher than for the other aptamers that were assayed, and, in some instances, the aptamer was able to access the interior of cells (e.g. kidney cells).

While not intending to be bound by theory, it is speculated that prolonged residence in the blood stream increases exposure of conjugated aptamer to tissues, leading to enhanced uptake, which is most pronounced in the case of highly perfused organs and in the case of inflamed tissues. The presence of aptamer in residual blood may contribute to, but is unlikely to account entirely for, the increased levels of the 20 kDa aptamer conjugate in perfused organs and inflamed tissue shown herein. The enhanced distribution of PEGylated aptamer to perfused organs and inflamed tissues represents extravasation, as suggested by experiments in mice dosed with tritiated 20 kDa PEG conjugate where [³H] signal was seen in cells of both the liver and kidney (See Examples provided below). Early work on aptamer therapeutics focused primarily on development of aptamers complexed with higher molecular weight (40 kDa) PEG species in an effort to avoid renal filtration (Reyderman and Stavchansky (1998), Pharmaceutical Research 15(6): 904-10; Tucker et al., (1999), J. Chromatography B. 732: 203-212; Watson, et al. (2000), Antisense Nucleic Acid Drug Dev. 10(2): 63-75; Carrasquillo, et al. (2003), Invest. Ophthalmology Vis. Sci. 44(1): 290-9). The present invention indicates that complexation with a smaller, e.g., 20 kDa, PEG polymer sufficiently protects aptamer-based drugs from renal filtration for many therapeutic indications. Smaller PEGs (e.g., 10 kDa to 20 kDa PEG moieties) also provide the collateral benefits of ease of synthesis and reduced cost of goods, as compared to larger PEGs.

PEG-Derivatized Nucleic Acids

Derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamer compositions of the invention may be derivatized with polyalkylene glycol (PAG) moieties. Examples of PAG-derivatized nucleic acids are found in U.S. patent application Ser. No. 10/718,833, filed on Nov. 21, 2003, which is herein incorporated by reference in its entirety. Typical polymers used in the invention include poly(ethylene glycol) (PEG), also known as or poly(ethylene oxide) (PEO) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpoly(ethylene glycol), can also be represented as HO-PEG-OH, where it is understood that the -PEG- symbol represents the following structural unit: —CH₂CH₂O—(CH₂CH₂O)ₙ—CH₂CH₂— where n typically ranges from about 4 to about 10,000.

As shown, the PEG molecule is di-functional and is sometimes referred to as “PEG diol.” The terminal portions of the PEG molecule are relatively non-reactive hydroxyl moieties, the —OH groups, that can be activated, or converted to functional moieties, for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively nonreactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics which generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with non-reactive methoxy end moiety, —OCH₃. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J. Org. Chem., 60:331-336 (1995). PAG conjugates are often used not only to enhance solubility and stability but also to prolong the blood circulation half-life of molecules.

Polyalkylated compounds of the invention are typically between 5 and 80 kD in size. Other PAG compounds of the invention are between 10 and 80 kD in size. Still other PAG compounds of the invention are between 10 and 60 kD in size. For example, a PAG polymer may be at least 10, 20, 30, 40, 50, 60, or 80 kD in size. Such polymers can be linear or branched.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa (further increases have minimal beneficial effect as half life becomes defined by macrophage-mediated metabolism rather than elimination via the kidneys).

Production of high molecular weight PEGs (>10 kDa) can be difficult, inefficient, and expensive. As a route towards the synthesis of high molecular weight PEG-nucleic acid conjugates, previous work has been focused towards the generation of higher molecular weight activated PEGs. One method for generating such molecules involves the formation of a branched activated PEG in which two or more PEGs are attached to a central core carrying the activated group. The terminal portions of these higher molecular weight PEG molecules, i.e., the relatively non-reactive hydroxyl (—OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol.2: 214-221, 2003).

The present invention provides another cost effective route to the synthesis of high molecular weight PEG-nucleic acid (preferably, aptamer) conjugates including multiply PEGylated nucleic acids. The present invention also encompasses PEG-linked multimeric oligonucleotides, e.g., dimerized aptamers. The present invention also relates to high molecular weight compositions where a PEG stabilizing moiety is a linker which separates different portions of an aptamer, e.g., the PEG is conjugated within a single aptamer sequence, such that the linear arrangement of the high molecular weight aptamer composition is, e.g., nucleic acid -PEG- nucleic acid -PEG- nucleic acid.

High molecular weight compositions of the invention include those having a molecular weight of at least 10 kD. Compositions typically have a molecular weight between 10 and 80 kD in size. High molecular weight compositions of the invention are at least 10, 20, 30, 40, 50, 60, or 80 kD in size.

A stabilizing moiety is a molecule, or portion of a molecule, which improves pharmacokinetic and pharmacodynamic properties of the high molecular weight aptamer compositions of the invention. In some cases, a stabilizing moiety is a molecule or portion of a molecule which brings two or more aptamers, or aptamer domains, into proximity, or provides decreased overall rotational freedom of the high molecular weight aptamer compositions of the invention. A stabilizing moiety can be a polyalkylene glycol, such a polyethylene glycol, which can be linear or branched, a homopolymer or a heteropolymer. Other stabilizing moieties include polymers such as peptide nucleic acids (PNA). Oligonucleotides can also be stabilizing moieties; such oligonucleotides can include modified nucleotides, and/or modified linkages, such as phosphorthioates. A stabilizing moiety can be an integral part of an aptamer composition, i.e., it is covalently bonded to the aptamer.

Compositions of the invention include high molecular weight aptamer compositions in which two or more nucleic acid moieties are covalently conjugated to at least one polyalkylene glycol moiety. The polyalkylene glycol moieties serve as stabilizing moieties. In compositions where a polyalkylene glycol moiety is covalently bound at either end to an aptamer, such that the polyalkylene glycol joins the nucleic acid moieties together in one molecule, the polyalkylene glycol is said to be a linking moiety. In such compositions, the primary structure of the covalent molecule includes the linear arrangement nucleic acid-PAG-nucleic acid. One example is a composition having the primary structure nucleic acid-PEG-nucleic acid. Another example is a linear arrangement of: nucleic acid -PEG- nucleic acid -PEG- nucleic acid.

To produce the nucleic acid—PEG—nucleic acid conjugate, the nucleic acid is originally synthesized such that it bears a single reactive site (e.g., it is mono-activated). In a preferred embodiment, this reactive site is an amino group introduced at the 5′-terminus by addition of a modifier phosphoramidite as the last step in solid phase synthesis of the oligonucleotide. Following deprotection and purification of the modified oligonucleotide, it is reconstituted at high concentration in a solution that minimizes spontaneous hydrolysis of the activated PEG. In a preferred embodiment, the concentration of oligonucleotide is 1 mM and the reconstituted solution contains 200 mM NaHCO₃-buffer, pH 8.3. Synthesis of the conjugate is initiated by slow, step-wise addition of highly purified bi-functional PEG. In a preferred embodiment, the PEG diol is activated at both ends (bi-activated) by derivatization with succinimidyl propionate. Following reaction, the PEG-nucleic acid conjugate is purified by gel electrophoresis or liquid chromatography to separate fully-, partially-, and un-conjugated species. Multiple PAG molecules concatenated (e.g., as random or block copolymers) or smaller PAG chains can be linked to achieve various lengths (or molecular weights). Non-PAG linkers can be used between PAG chains of varying lengths.

The 2′-O-methyl, 2′-fluoro modifications stabilize the aptamer against nucleases and increase its half life in vivo. The 3′-3′-dT cap also increases exonuclease resistance. See, e.g., U.S. Pat. Nos. 5,674,685; 5,668,264; 6,207,816; and 6,229,002, each of which is incorporated by reference herein in its entirety.

PAG-Derivatization of a Reactive Nucleic Acid

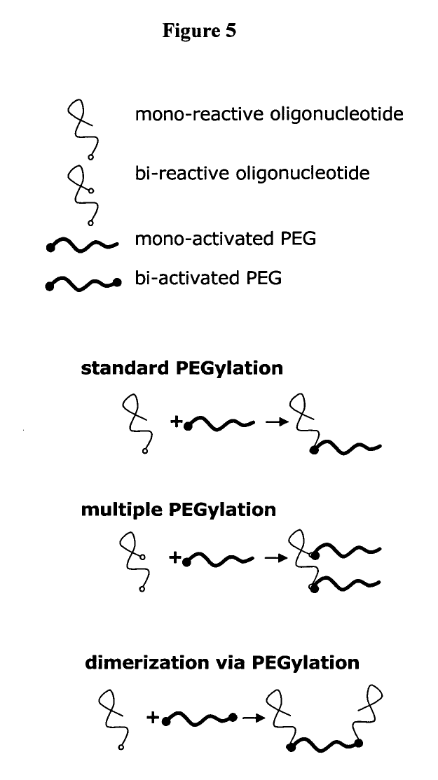

High molecular weight PAG-nucleic acid-PAG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation as illustrated in FIG. 5. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully-, partially-, and un-conjugated species.

The linking domains can also have one ore more polyalkylene glycol moieties attached thereto. Such PAGs can be of varying lengths and may be used in appropriate combinations to achieve the desired molecular weight of the composition.

The effect of a particular linker can be influenced by both its chemical composition and length. A linker that is too long, too short, or forms unfavorable steric and/or ionic interactions with the target will preclude the formation of complex between aptamer and target. A linker, which is longer than necessary to span the distance between nucleic acids may reduce binding stability by diminishing the effective concentration of the ligand. Thus, it is often necessary to optimize linker compositions and lengths in order to maximize the affinity of an aptamer to a target.

Nucleic Acid Sensor Molecules (NASMs)

Nucleic acid sensor molecules are nucleic acid molecules (e.g., DNA or RNA molecules) that include a target recognition domain, a catalytic domain, and, optionally, a linker domain connecting the catalytic domain. Thus, NASMs include allosteric ribozymes, whose activity is switched on or off by the presence of a specific target. Allosteric ribozymes can act as reporter molecules in that they directly couple molecular detection to the triggering of a chemical reaction. Because they are also target molecule specific, however, they can also be used in much the same way as aptamers, e.g., to deliver toxins to a target. The combination of these properties in a single molecule makes them powerful tools for a wide range of applications.

Nucleic acid sensor molecules suitable for use in the compositions and methods of the invention are disclosed in, e.g., WO 03/014375 which is incorporated herein by reference.

Nucleic acid-based detection schemes have exploited the ligand-sensitive catalytic properties of some nucleic acids, e.g., such as ribozymes. Ribozyme-based nucleic acid sensor molecules have been designed both by engineering and by in vitro selection methods. Some engineering methods exploit the apparently modular nature of nucleic acid structures by coupling molecular recognition to signaling by simply joining individual target-modulation and catalytic domains using, e.g., a double-stranded or partially double-stranded linker. ATP sensors, for example, have been created by appending the previously-selected, ATP-selective sequences (see, e.g., Sassanfar et al., Nature 363:550-553 (1993)) to either the self-cleaving hammerhead ribozyme (see, e.g., Tang et al., Chem. Biol. 4:453-459 (1997)) as a hammerhead-derived sensor, or the LI self-ligating ribozyme (see, e.g., Robertson et al., Nucleic Acids Res. 28:1751-1759 (2000)) as a ligase-derived sensor. Hairpin-derived sensors are also contemplated. In general, the target modulation domain is defined by the minimum number of nucleotides sufficient to create a three-dimensional structure which recognizes a target molecule.

Catalytic nucleic acid sensor molecules (NASMs) are selected which have a target molecule-sensitive catalytic activity (e.g., self-cleavage) from a pool of randomized or partially randomized oligonucleotides. The catalytic NASMs have a target modulation domain which recognizes the target molecule and a catalytic domain for mediating a catalytic reaction induced by the target modulation domain's recognition of the target molecule. Recognition of a target molecule by the target modulation domain triggers a conformational change and/or change in catalytic activity in the nucleic acid sensor molecule. In one embodiment, by modifying (e.g., removing) at least a portion of the catalytic domain and coupling it to an optical signal generating unit, an optical nucleic acid sensor molecule is generated whose optical properties change upon recognition of the target molecule by the target modulation domain. In one embodiment, the pool of randomized oligonucleotides comprises the catalytic site of a ribozyme.

A heterogeneous population of oligonucleotide molecules comprising randomized sequences is screened to identify a nucleic acid sensor molecule having a catalytic activity which is modified (e.g., activated) upon interaction with a target molecule. As with the aptamer nucleic acids, the oligonucleotide can be RNA, DNA, or mixed RNA/DNA, and can include modified or normatural nucleotides or nucleotide analogs.

Each oligonucleotide in the population comprises a random sequence and at least one fixed sequence at its 5′ and/or 3′ end. In one embodiment, the population comprises oligonucleotides which include as fixed sequences an aptamer known to specifically bind a particular target and a catalytic ribozyme or the catalytic site of a ribozyme, linked by a randomized oligonucleotide sequence. In a preferred embodiment, the fixed sequence comprises at least a portion of a catalytic site of an oligonucleotide molecule (e.g., a ribozyme) capable of catalyzing a chemical reaction.

Catalytic sites are well known in the art and include, e.g., the catalytic core of a hammerhead ribozyme (see, e.g., U.S. Pat. No. 5,767,263; U.S. Pat. No. 5,700,923) or a hairpin ribozyme (see, e.g., U.S. Pat. No. 5,631,359). Other catalytic sites are disclosed in U.S. Pat. No. 6,063,566; Koizumi et al., FEBS Lett. 239: 285-288 (1988); Haseloff and Gerlach, Nature 334: 585-59 (1988); Hampel and Tritz, Biochemistry 28: 4929-4933 (1989); Uhlenbeck, Nature 328: 596-600 (1987); and Fedor and Uhlenbeck, Proc. Natl. Acad. Sci. USA 87: 1668-1672 (1990).

In some embodiments, a population of partially randomized oligonucleotides is generated from known aptamer and ribozyme sequences joined by the randomized oligonucleotides. Most molecules in this pool are non-functional, but a handful will respond to a given target and be useful as nucleic acid sensor molecules. Catalytic NASMs are isolated by the iterative process described above. In all embodiments, during amplification, random mutations can be introduced into the copied molecules—this ‘genetic noise’ allows functional NASMs to continuously evolve and become even better adapted as target-activated molecules.

In another embodiment, the population comprises oligonucleotides which include a randomized oligonucleotide linked to a fixed sequence which is a catalytic ribozyme, the catalytic site of a ribozyme or at least a portion of a catalytic site of an oligonucleotide molecule (e.g., a ribozyme) capable of catalyzing a chemical reaction. The starting population of oligonucleotides is then screened in multiple rounds (or cycles) of selection for those molecules exhibiting catalytic activity or enhanced catalytic activity upon recognition of the target molecule as compared to the activity in the presence of other molecules, or in the absence of the target.

The nucleic acid sensor molecules identified through in vitro selection, e.g., as described above, comprise a catalytic domain (i.e., a signal generating moiety), coupled to a target modulation domain, (ie., a domain which recognizes a target molecule and which transduces that molecular recognition event into the generation of a detectable signal). In addition, the nucleic acid sensor molecules of the present invention use the energy of molecular recognition to modulate the catalytic or conformational properties of the nucleic acid sensor molecule.

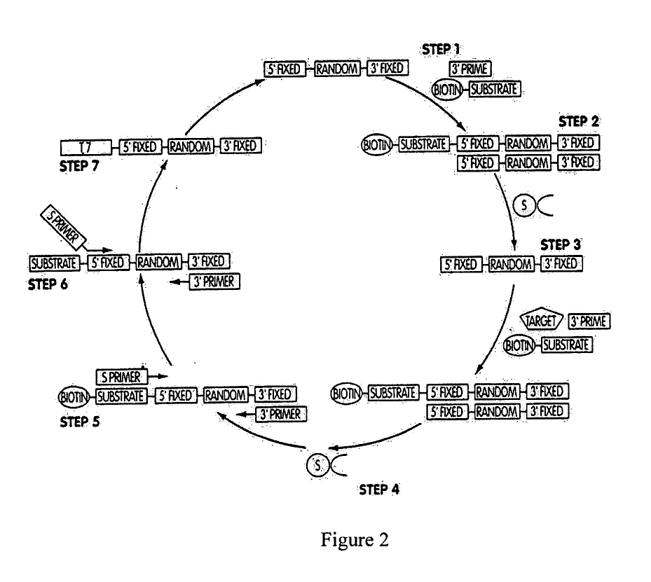

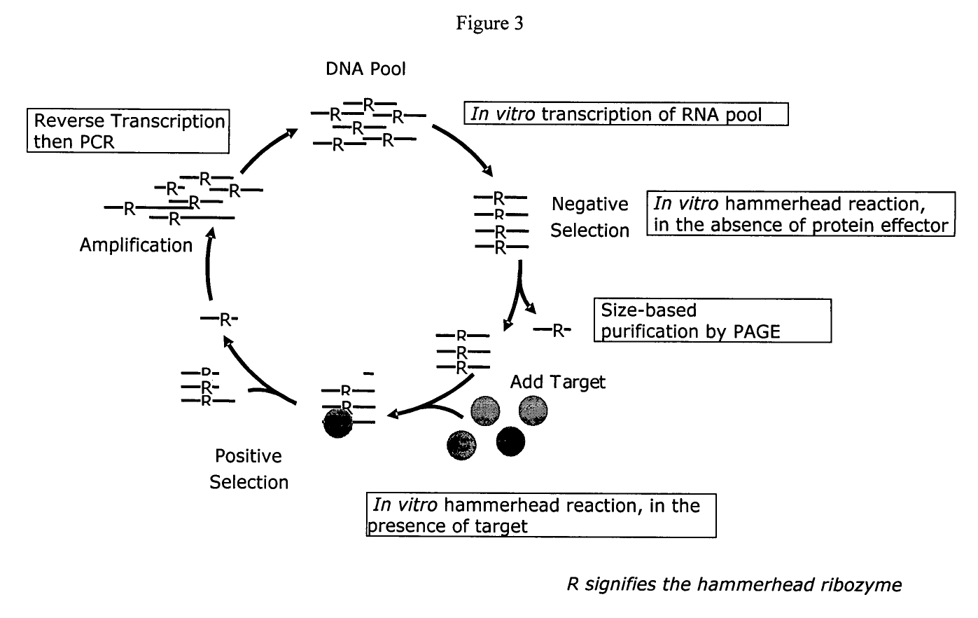

Nucleic acid sensor molecules are generally selected in a 5 to 20 cycle procedure. In one embodiment, heterogeneity is introduced only in the initial selection stages and does not occur throughout the replicating process. FIG. 2 shows a schematic diagram in which the oligonucleotide population is screened for a nucleic acid sensor molecule which comprises a target molecule activatable ligase activity. FIG. 3 shows the hammerhead nucleic acid sensor molecule selection methodology. Each of these methods are readily modified for the selection of NASMs with other catalytic activities.

Additional procedures may be incorporated in the various selection schemes, including: pre-screening, negative selection, etc. For example, individual clones isolated from selection experiments are tested early for allosteric activation in the presence of target-depleted extracts as a pre-screen, and molecules that respond to endogenous non-specific activators are eliminated from further consideration as target-modulated NASMs; to the extent that all isolated NASMs are activated by target-depleted extracts, depleted extracts are included in a negative selection step of the selection process; commercially available RNase inhibitors and competing RNAse substrates (e.g., tRNA) may be added to test samples to inhibit nucleases; or by carrying out selection in the presence of nucleases (e.g., by including depleted extracts during a negative selection step) the experiment intrinsically favors those molecules that are resistant to degradation; covalent modifications to RNA that can render it highly nuclease-resistant can be performed (e.g., 2′-O-methylation) to minimize non-specific cleavage in the presence of biological samples (see, e.g., Usman et al.). Clin. Invest. 106:1197-202 (2000).

In one embodiment, nucleic acid sensor molecules are selected which are activated by target molecules comprising molecules having an identified biological activity (e.g., a known enzymatic activity, receptor activity, or a known structural role); however, in another embodiment, the biological activity of at least one of the target molecules is unknown (e.g., the target molecule is a polypeptide expressed from the open reading frame of an EST sequence, or is an uncharacterized polypeptide synthesized based on a predicted open reading frame, or is a purified or semi-purified protein whose function is unknown).

Although in one embodiment the target molecule does not naturally bind to nucleic acids, in another embodiment, the target molecule does bind in a sequence specific or non-specific manner to a nucleic acid ligand. In a further embodiment, a plurality of target molecules binds to the nucleic acid sensor molecule. Selection for NASMs specifically responsive to a plurality of target molecules (i.e., not activated by single targets within the plurality) may be achieved by including at least two negative selection steps in which subsets of the target molecules are provided. Nucleic acid sensor molecules can be selected which bind specifically to a modified target molecule but which do not bind to closely related target molecules. Stereochemically distinct species of a molecules can also be targeted.

Toxins

Toxins useful in the present invention include chemotoxins having cytotoxic effects. These can be classified in their mode of action: 1) tubulin stabilizers/destabilizers; 2) anti-metabolites; 3) purine synthesis inhibitors; 4) nucleoside analogs; and 5) DNA alkylating or modifying agents. Radioisotopes also have cytotoxic effects useful in the present invention.

Examples of suitable toxins include, e.g., chemotherapeutic agents. Chemotherapeutics are typically small chemical entities produced by chemical synthesis and include cytotoxic drugs, cytostatic drugs as well as compounds which affect cells in other ways such as reversal of the transformed state to a differentiated state or those which inhibit cell replication. Examples of chemotherapeutics include, but are not limited to: methotrexate (amethopterin), doxorubicin (adrimycin), daunorubicin, cytosinarabinoside, etoposide, 5-4 fluorouracil, melphalan, chlorambucil, and other nitrogen mustards (e.g., cyclophosphamide), cis-platinum, vindesine (and other vinca alkaloids), mitomycin and bleomycin.

Toxins can include complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin. Protein toxins may be produced using recombinant DNA techniques as fusion proteins which include peptides of the invention. Protein toxins may also be conjugated to compounds of the invention by non-peptidyl bonds. In addition, photosensitizers and cytokines can also be used with the present invention.

Cytotoxic molecules that can be used in the present invention are anthracycline family of cytotoxic agents, e.g., doxorubicin (DOX). Doxorubicin damages DNA by intercalation of anthracycline protion, metal ion, chelation, or by generation of free radicals. DOX has also been shown to inhibit DNA topoisomerase II. Doxorubicin has been shown clinically to have broad spectrum of activity and toxic side effects that are both dose-related and predictable. Efficacy of DOX is limited by myelosuppression and cardiotoxicity. Complexed with a targeting moiety such as an aptamer increases intratumoral accumulation while reducing systemic exposure.

Maytansinoids are very toxic chemotherapeutic molecules that can be used as therapeutic moieties of the present invention. Maytansinoids effect their cytotoxicity by inhibiting tubulin polymerization, thus inhibiting cell division and proliferation. Maytansinoid derivative DM1 has been conjugated to other targeting moieties, e.g., murine IgG1 mAb against MUC-1 and to an internalizing anti-PSMA murine monoclonal antibody 8D11 (mAb) through disulfide linker chemistry.

Enediynes are another class of cytotoxic molecules that can be used as therapeutic moieties of the present invention. Enediynes effect their cytotoxicity by producing double-stranded DNA breaks at very low drug concentrations. The enediynes class of compounds includes calicheamicins, neocarzinostatin, esperamicins, dynemicins, kedarcidin, and maduropeptin. Linking chemistries for these compounds include periodate oxidation of carbohydrate residues followed by reaction with a hydrazide derivative of calicheamycin, for example. These conjugates utilize an acid-labile hydrazone bond to a targeting moiety, such as a monoclonal antibody to ensure hydrolysis following internalization into lysosomes, and a sterically protected disulfide bond to calicheamicin to increase stability in circulation.

Tumor therapeutics also include radionuclides, particularly high energy alpha particle emitters. Alpha particles are high energy, high linear energy transfer (LET) helium nuclei capable of strong, yet selective cytotoxicity. Approximately 100 radionuclides decay with alpha emission. A single atom emitting an alpha particle can have a lethal cytotoxic effect on a single cell. Conjugates of radionuclides to mAbs have been used in preclinical models of leukemia and prostate cancer, and a phase I clinical trial is underway with ²¹¹At-labeled anti tenascin mAb against malignant gliomas.

Radioisotopes may be conjugated to compounds of the invention. Examples of radioisotopes which are useful in radiation therapy include, e.g., ⁴⁷SC, ⁶⁷CU, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi. Some alpha particle emitting radioisotopes exhibit too short a half life to be effective therapeutics against most tumors. For example, ²¹³Bi has a 46 minute half life which limits its efficacy to only the most accessible cancer cells, and poses practical obstacles such as timely shipment and administration. Another radioisotope ²²⁵Ac is a more suitable radiotherapeutic because each ²²⁵Ac atom decays into several daughter atoms, four of which also emits alpha particles.

Nucleic Acid-Drug Conjugates

The present invention provides materials and methods to produce therapeutic nucleic acid-drug conjugates. These conjugates are described the following general formula: (nucleic acid sequence)ₙ—linker—(drug)ₘ, wherein n is between 1 and 10 and m is between 0 and 20. In one embodiment, the conjugates are aptamer-drug conjugates that have the following general formula: (aptamer)ₙ—linker—(drug)ₘ, wherein n is between 1 and 10 and m is between 0 and 20. In this embodiment, a plurality of aptamer sequences and drug species, e.g., toxins, may be combined to yield a therapeutic composition. For example, the conjugate contains a mixture of a first and second aptamer species that are specific for two different therapeutic targets (e.g. an anti-VEGF₁₆₅ aptamer and an anti-PDGF-BB aptamer), such that the first and second aptamer sequences are linked to a single linker-drug conjugate.

The invention also provides methods of using these therapeutic aptamer-drug conjugates in an improved method for the targeted delivery of drugs by exploiting the intrinsic binding specificity of aptamers. Additionally, these therapeutic aptamer-drug conjugates provide a means for improving the pharmacokinetic properties of aptamers and drugs, thereby increasing their in vivo half-life and altering their biodistribution.

Specific examples of therapeutic aptamer-drug conjugates of the invention and methods of generating these conjugates are described in Example 5. Attachment of nucleic acids (aptamers and/or NASMs) to toxins

The present invention provides materials and methods to produce bifunctional molecules that consist of a targeting moiety that localizes to target cells, e.g., tumor cells, or neovasculature, said targeting molecule coupled with a therapeutic moiety that effects a cytotoxic effect on the target cells. The present invention provides nucleic acid targeting moieties and therapeutic agents, for example cytotoxic agents (small organic molecules), radionuclides, plant and bacterial toxins, enzymes, photosensitizers, and cytokines.

Nucleic acid targeting moieties of the present invention can be attached to therapeutic moieties, e.g., toxins, using methods known in the art. For example, methods for generating blended nucleic acid ligands comprised of functional unit(s) added to provide a nucleic acid ligand with additional functions are described in U.S. Pat. No. 5,683,867, U.S. Pat. No. 6,083,696, and U.S. Pat. No. 5,705,337. The latter patent discloses methods for identifying nucleic acid ligands capable of covalently interacting with targets of interest. The nucleic acids can be associated with various functional units. The method also allows for the identification of nucleic acids that have facilitating activities as measured by their ability to facilitate formation of a covalent bond between the nucleic acid, including its associated functional unit, and its target.

Cytotoxics—Small Organic Molecule Linking Chemistries

To link nucleic acid aptamers of the present invention to small molecule cytotoxic agents that contain carboxylate groups, the latter are converted into an amine-reactive probe (e.g. NHS ester) by conventional synthetic organic reactions, and then coupled to an amine oligonucleotide aptamer. Amine-containing small molecules can be coupled to an activated oligo (e.g. 5′-carboxy-modifier C10 (Glen Research) according to the Glen technical product bulletin). Alternatively, an amine-oligo can be activated in situ by crosslinking reagents, including but not limited to DSS, BS³ or related reagents (Pierce, Rockford, Ill.), and further coupled to amines.

Thiol-containing small molecules can be coupled to 2,2-dithio-bispyridine activated thiol aptamer or an SPDP-activated (Pierce, Rockford, Ill.) amine-oligo.

Small molecules that do not contain carboxylate, amine or thiol groups are preferably converted into such by conventional synthetic organic chemistry by methods known to those of skill in the art.

Additionally, encapsulated (e.g. in liposomes) cytotoxics can also be linked to aptamers or NASMs of the present invention with acid-labile linkers, enzyme cleavable linkers used in the art for linking liposome to reactive moieties, such as activated oligonucleotides.

Acid-labile linkers include for illustration but not limitation, cis-aconityl linkers used to link anthracyclines, doxorubicin (DOX) or daunorubicin (DNR), to immunoconjugates such as several mAbs (e.g., anti-melanoma mAb 9.927); leading to released cytotoxic agents in the environment of lysozomes.

Hydrazone linkers have been used to conjugate small molecule cytotoxic agents including DNR, morpholino-DOX to anti-αvβ3 mAb LM609, and anti-Le^{y} mAb BR96. These hydrazone linkers are acid labile at pH 4.5. Other acid-sensitive anthracycline conjugates have been obtained through modification of the C-13 carbonyl group to give acylhydrazone, semicarbazones, thiosemicarbazones and oximes.

Cytotoxics—Peptides (Synthetic) Linking Chemistries

In the case of peptide cytotoxic agents, methods for coupling of synthetic peptides include synthesis of an amine-reactive activated ester (e.g., NHS) of the peptide, coupling to amine-oligo.

Another method of linking peptide cytotoxic moieties to the targeting moieties of the present invention also include synthesis of a cytotoxic peptide moiety with an extra C- or N-terminal cysteine. This can be activated with 2,2-dithio-bispyridine and coupled to a thiol-modified aptamer oligo (standard automated synthesis, final coupling with an thiol-modifier [Glen Research, Sterling, Va.]). Alternatively, the thiol-modified aptamer is activated with 2,2-dithio-bispyridine and coupled to the cys-peptide. Lastly, an amino-terminated oligo can be activated with SPDP (Pierce, Rockford, Ill.) and coupled to the cys-containing peptide. All three methods generate the conjugate coupled through a disulfide bond.

Another method of linking peptide cytotoxic moieties to the targeting moieties of the present invention also includes modification of a targeting moiety consisting of an amine-oligo with a maleimide reagent, e.g., GMBS, (Pierce, Rockford, Ill.), subsequent coupling to cys-peptide.

Another method of linking peptide cytotoxic moieties to the targeting moieties of the present invention also includes synthesis of a targeting moiety consisting of an oligo modified with 5′-carboxy-modifier C10 (Glen Research) and in-situ coupling to an amine-containing molecule (i.e. peptide) according to methods known in the art.

Another method of linking peptide cytotoxic moieties to the targeting moieties of the present invention also includes oxidizing 3′-ribo-terminated oligos with sodium meta-periodate and the resulting aldehyde reacted with amine peptides in the presence of reducing agents. In addition, C-terminal peptide hydrazides can couple to an oxidized RNA even without the aid of reducing agents.

Cytotoxics—Protein Linking Chemistries

Methods of linking cytotoxic protein moieties of the present invention to targeting moieties of the present invention are principally the same as those methods used for linking peptides.

Methods of linking protein cytotoxic protein moieties of the present invention include activation of the targeting moiety of the invention consisting of an amino-terminated oligo with e.g. SPDP or GMBS (Pierce, Rockford, Ill.), or of an thiol-oligo with 2,2-dithio-bispyridine and coupling to the cys-containing protein.

Another method of linking cytotoxic protein moieties of the invention with targeting moieties of the present invention include coupling of protein amines to an amine-containing oligo using crosslinking reagents, e.g., DSS, BS³ or related reagents (Pierce, Rockford, Ill.).

Radioisotopes Cytotoxic Moieties Linking Chemistries

Methods of linking cytotoxic moieties of the present invention consisting of radioactive metal ions (e.g., isotopes of Tc, Y, Bi, Ac, Cu etc.) to targeting moieties of the present invention include chelation with a suitable ligand, such as DOTA (Lewis, et al., Bioconjugate Chemistry 2002, 13, 1178). A generic labeling scheme would start with the synthesis of a 5′-amino-modified aptamer oligo (standard automated synthesis, final coupling with an amino-modifier [Glen Research, Sterling, Va.]). Then, the chelator is converted into an amine-reactive activated ester, and subsequently coupled to the oligo similar to the method described in Lewis, et al.

Another method of linking radionuclide cytotoxic moieties of the present invention to targeting moieties of the present invention include oxidizing 3′-ribo-terminated oligos with sodium meta-periodate and the resulting aldehyde reacted with amine-containing chelators or radiolabels in the presence of reducing agents. Alternatively, hydrazine, hydrazide, semicarbazide and thiosemicarbazide derivatives of chelators or radiolabels can be used.

Additional methods for attaching nucleic acids to non-nucleic acid molecules are disclosed in, e.g., WO 00/70329. The publication discloses compositions, systems, and methods for simultaneously detecting the presence and quantity of one or more different compounds in a sample using aptamer beacons. Aptamer beacons are oligonucleotides that have a binding region that can bind to a non-nucleotide target molecule, such as a protein, a steroid, or an inorganic molecule. New aptamer beacons having binding regions configured to bind to different target molecules can be used in solution-based and solid, array-based systems. The aptamer beacons can be attached to solid supports, e.g., at different predetermined points in two-dimensional arrays.

Pharmaceutical Compositions

The invention also includes pharmaceutical compositions containing aptamer-toxin molecules. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. Compositions of the invention are useful for administration to a subject suffering from, or predisposed to, a disease or disorder which is related to or derived from a target to which the aptamers specifically bind.

For example, the target is a protein involved with a pathology, for example, the target protein causes the pathology.

Compositions of the invention can be used in a method for treating a patient or subject having a pathology. The method involves administering to the patient or subject a composition comprising aptamers that bind a target (e.g., a protein) involved with the pathology, so that binding of the composition to the target alters the biological function of the target, thereby treating the pathology.

The patient or subject having a pathology, e.g. the patient or subject treated by the methods of this invention can be a mammal, or more particularly, a human.

One aspect of the invention comprises an aptamer composition of the invention in combination with other treatments for cytokine related disorders. The aptamer composition of the invention may contain, for example, more than one aptamer. In some examples, an aptamer composition of the invention, containing one or more compounds of the invention, is administered in combination with another useful composition such as an anti-inflammatory agent, an immunosuppressant, an antiviral agent, or the like. Furthermore, the compounds of the invention may be administered in combination with a chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

“Combination therapy” (or “co-therapy”) includes the administration of an aptamer composition of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical. “Combination therapy” also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The compounds of the invention and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

In practice, the compounds or their pharmaceutically acceptable salts, are administered in amounts which will be sufficient to induce lysis of a desired cell.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer-toxin and/or NASM molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 5000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

The foregoing being a detailed description of the present invention, persons of skill in the art will understand the following examples to be illustrative of embodiments of aspects of the present invention. Persons of skill in the art will also understand that the foregoing examples are for illustration of the present invention and not limitation thereof. Accordingly, the invention is to be defined not by the preceding illustrative description but instead by the spirit and scope of the claims that follow.

### EXAMPLE 1

### PDGF Aptamer—⁹⁰Y Conjugate

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamers are described in U.S. Pat. No. 5,723,594 incorporated by reference herein. The aptamer is synthesized according to standard methods known to those skilled in the art including phosphoramidite synthesis methods so that an amine terminus is present on the aptamer. The amine derivatized aptamer is then conjugated to a1,4,7,10-tetraazacyclododecane-,N,N,N′,N″-tetraacetic acid (DOTA) linker reagent and the ⁹⁰Y isotope is chelated to the derivatized DOTA-aptamer complex according to Lewis, et al., Bioconjugate Chemistry, 2001, 12, 320-324.

The apatamer-⁹⁰Y conjugate is then administered to the subject or patient in a therapeutically effective amount to inhibit the disease state in the subject or patient.

### EXAMPLE 2

### PDGF Aptamer—ArinA Peptide Conjugates

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamer are described in U.S. Pat. No. 5,723,594 incorporated by reference herein. The aptamer is synthesized according to methods know to those skilled in the art including phosphoramidite synthesis. The last coupling in the oligonucleotide synthesis is done using a OPeC™ reagent phosphoramidite (Glen Research, Sterling, Va.). This is done according to the following method by Stetsenko et al., New phosphoramidite reagents for the synthesis of oligonucleotides containing a cysteine residue useful in peptide conjugation, Nucl. Acids (2000) 19, 1751-1764. The cytotoxic peptide is synthesized according to standard methods using the Pentafluorophenyl S-benzylthiosuccinate, Peptide Modifying Reagent (PMR) reagent in the final coupling step in standard Fmoc-based solid-phase peptide assembly. The conjugation of the reactive aptamer and the arinA cytotoxic peptide is done by methods described in Stetsenko, et al.

Once an aptamer-peptide conjugate has been synthesized, the therapeutic conjugate is administered to a subject or patient in a therapeutically effective amount to treat the disease state in the subject or patient. The PDGF aptamer targeting moiety brings the cytotoxic peptide in close proximity to the target cell and the peptide exerts its cytotoxic effect on the cell having a PDGF marker.

### EXAMPLE 3

### PDGF Aptamer—Protein Conjugate

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamers are described in U.S. Pat. No. 5,723,594 incorporated by reference herein. The aptamer is synthesized according to methods know to those skilled in the art including phosphoramidite synthesis and so that a thiol from a cysteine reactive terminus is present in the modified aptamer to be linked. This is done according to the method by Tung, et al., Bioconjugate Chemistry, 2000, 11, 605-618. The cysteine derivatized aptamer is then conjugated to the cytotoxic protein by a peptide modifying reagent linker having a reactive group that forms a covalent bond with the —SH reactive end of the modified oligo. This results in an oligonucleotide-peptide conjugate as described by Tung, et al.

Once the therapeutic conjugate is synthesized, it is administered to a subject or patient in a therapeutically effective amount to treat the disease state in the subject or patient. The PDGF aptamer targeting moiety brings the cytotoxic protein in close proximity to the target cell and the protein exerts its cytotoxic effect on the cell having a PDGF marker.

### EXAMPLE 4

### PDGF Aptamer—DNR/DOX Chemotoxic Organic Molecule Conjugate

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamers are described in U.S. Pat. No. 5,723,594 incorporated by reference herein. The aptamer is synthesized according to methods know to those skilled in the art including hydrazidephosphoramidite synthesis so that a carbonyl reactive terminus is present. This is done according to the following method by Raddatz, et al., Hydrazide oligonucleotides: new chemical modification for chip array attachment and conjugation. Nucleic Acids Res., 2002 Nov 1:30(21):4793-802. The hydrazide derivatized aptamer is then conjugated to the carbonyl functional group of the DOX or DNR chemotoxic organic molecule according to Trail, et al., Cancer Immunol Immunother, (2003) 52:328-337, and references cited therein.

Once the PDGF aptamer-DOX or DNR conjugate is created it is administered to the subject or patient having a proliferative disease where PDGF is a marker and is involved in its pathogenesis. Once the DOX/DNR is brought in close proximity of the target cell by the PDGF specific aptamer, the DOX/DNR cytotoxic moiety exerts its cytotoxic effect on the targeted cells reducing non-specific collateral damage to non-target cells or surrounding tissue.

### EXAMPLE 5

### Therapeutic Aptamer-Drug Conjugates

As described above, the therapeutic aptamer-drug conjugates of the invention have the following general formula: (aptamer)ₙ—linker—(drug)ₘ, where n is between 1 and 10 and m is between 0 and 20. A plurality of aptamer species and drug species may be combined to yield a therapeutic composition.

In one embodiment, the therapeutic aptamer-drug conjugates of the invention are used in the targeted killing of tumor cells through aptamer-mediated delivery of cytotoxins. The efficiency of cell killing is improved if the target tumor marker is a marker that readily internalizes or recycles into the tumor cell.

Tumor Cell-Targeting Aptamers: In this particular embodiment of the invention, the aptamer used in the aptamer-drug conjugate is selected for the ability to specifically recognize a marker that is expressed preferentially on the surface of tumor cells, but is relatively deficient from all normal tissues. Suitable target tumor markers include, but are not limited to, those listed below in Table 1.

TABLE 1
Aptamer Targets for Cytotoxin Delivery to Tumor Cells

| | |
|---|---|
| PSMA | |
| PSCA | |
| E-selectin | |
| EphB2 (and other representative ephrins) | |
| Cripto-1 | |
| TENB2 (also known as TEMFF2) | |
| ERBB2 receptor (HER2) | |
| MUC1 | |
| CD44v6 | |
| CD6 | CD30 |
| CD19 | CD33 |
| CD20 | CD56 |
| CD22 | IL-2 receptor |
| CD23 | HLA-DR10β subunit |
| CD25 | |
| EGFRvIII | |
| MN antigen (also known as | |
| CA IX or G250 antigen) | |
| Caveolin-1 | |
| Nucleolin | |

Aptamers that are specific for a given tumor cell marker, such as those listed in Table 1, are generated using the SELEX™ process, as described above. SELEX™ has been successfully used to generate aptarners both to isolated, purified tumor cell surface proteins (e.g. tenascin C, MUC1, PSMA) and to tumor cells cultured in vitro (e.g. U251 (glioblastoma cell line), YPEN-1 (transformed prostate endothelial cell line)). In most cases, the extracellular portion of an identified tumor marker protein is recombinantly expressed, purified, and treated as a soluble protein through the SELEX process. In those cases where soluble protein domains cannot readily be produced, direct selection for binding to transformed cells (optionally negatively selecting against normal cell binding) yields aptamers that bind to tumor-specific markers.

Aptamer sequences initially identified through application of the SELEX process are optimized for both large-scale synthesis and in vivo applications through a progressive set of modifications. These modifications include, for example, (1) 5′- and 3′-terminal and internal deletions to reduce the size of the aptamer, (2) doped reselection for sequence modifications that increase the affinity or efficiency of target binding, (3) introduction of stabilizing base-pair changes that increase the stability of helical elements in the aptamer, (4) site-specific modifications of the 2′-ribose (e.g. 2′-hydroxyl ˜2′-O-methyl substitutions) and phosphate (e.g. phosphodiester→phosphorothioate substitutions) positions to both increase thermodynamic stability and to block nuclease attack in vivo, and (5) the addition of 5′- and/or 3′-caps (e.g. inverted 3′-deoxythymidine) to block attack by exonucleases. Aptamers generated through this process are typically 15-40 nucleotides long and exhibit serum half-lives greater than 10 hours.

To facilitate synthesis of the aptamer conjugate, reactive nucleophilic or electrophilic attachment points are introduced, for example, by directed solid phase synthesis or by post-synthesis modifications. A free amine is introduced at either the 5′- or 3′-end of the aptamer by incorporating the appropriate amino-modifier phosphoramidite at the end or beginning of solid phase synthesis respectively (e.g. 5′-amino modifier C6, Glen Research, VA; or 3′-PT-Amino-Modifier C6 CPG Glen Research, VA, respectively). This amine serves directly as a nucleophilic attachment point, or alternatively, this amine is further converted into an electrophilic attachment point. For example, reaction with bis(sulfosuccinimidyl) suberate (BS³) or related reagents (Pierce, Ill.) yields a NHS ester suitable for conjugation with amine containing molecules. Alternatively, carboxylic acid groups are introduced by using 5′-Carboxy Modifier C10 (Glen Research, VA) at the end of aptamer solid phase synthesis. Such carboxylates are then activated in situ with, e.g., 1-Ethyl-3-[3-dimethylaminopropyl]carbodiimide (EDC) to further react with nucleophiles.

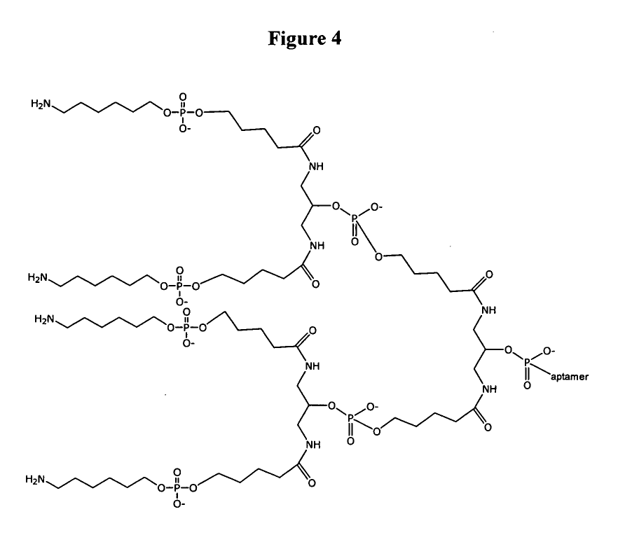

Multiple amines may be introduced at the 5′-end of the aptamer through solid phase synthesis in which a 5′-symmetric doubler is incorporated one or more times and followed with a terminal reaction with the 5′-amino modifier described above. Symmetric doubler phosphoramidites are commercially available (e.g. Glen Research, VA). As shown in FIG. 4, two rounds of coupling with the symmetric doubler followed by amine capping yield an aptamer bearing four free reactive amines.

Cytotoxins: Drugs are attached to the linker such that their pharmacological activity is preserved in the conjugate or such that in vivo metabolism of the conjugate leads to release of pharmacologically active drug fragments. Table 2 lists potent cytotoxins which are suitable for conjugation. Previous efforts to synthesize antibody conjugates or to generate pharmacologically active variants of these cytotoxins has, in some cases, provided useful insights into which functional groups are amenable to modification. The following modified cytotoxics may be used to construct aptamer-linker-drug conjugates.

Calicheamicins: N-acetyl gamma calicheamicin dimethyl hydrazide (NAc-γ-DMH) presents a reactive hydrazide group that readily reacts with aldehydes to form the corresponding hydrazone. NAc-γ-DMH can be used directly to conjugate to aldehyde bearing linkers, or, alternatively, can be converted to an N-hydroxysuccinimide-bearing amine-reactive form (NAc-γ-NHS) as described by Hamann et al. (Bioconjugate Chem., 13: 47-58 (2002)) to be conjugated to amine-bearing aptamers.

Maytansinoids: Conjugatable forms of maytansinoids are accessible through re-esterification of maytansinol which itself may be produced as described in U.S. Pat. Nos. 4,360,462 and 6,333,410 through reduction of maytansine or ansamitocin P-3 using one of several reducing agents (including lithium aluminum hydride, lithium trimethoxyaluminum hydride, lithium triethoxyaluminum hydride, lithium tripropoxyaluminum hydride, and the corresponding sodium salts). Maytansinol may subsequently be converted to an amine-reactive form as described in U.S. Pat. No. 5,208,020 by (1) reaction with a disulfide-containing carboxylic acid (e.g. the variety of linkers considered in U.S. Pat. No. 5,208,020) in the presence of carbodiimide (e.g. dicylcohexylcarbodiimide) and catalytic amounts of zinc chloride (as described in U.S. Pat. No. 4,137,230), (2) reduction of the disulfide using a thiol-specific reagent (e.g. dithiothreitol) followed by HPLC purification to yield a thiol-bearing maytansinoid, and (3) reaction with a bifunctional thiol- and amine-reactive crosslinking agent (e.g. N-succinimidyl 4-(2-pyridyldithio) pentanoate). A representative activated maytansinoid bearing an amine-reactive N-hydroxysuccinimide suitable for conjugate formation is shown in Table 2 (May-NHS).

Vinca alkaloids: Vinca alkaloids such as vinblastine can be conjugated directly to aldehyde-bearing linkers following conversion to a hydrazide form as described by Brady et al. (J. Med. Chem., 45:4706-4715, 2002). Briefly, vinblastine sulfate is dissolved in 1:1 hydrazine/ethanol and heated to 60° C.-65° C. for 22 hours to yield desacetylvinblastine 3-carboxhydrazide (Table 2, DAVCH). Alternatively, amine-reactive forms of vinblastine may be generated in situ as described by Trouet et al. (U.S. Pat. No. 4,870,162) by (1) initially converting vinblastine sulfate to the desacetyl form (e.g. as described by Brady et al., reacting with 1:3 hydrazine/methanol at 20° C. for 20 hours), (2) reacting the resulting free base with approximately 2-fold excess succinic anhydride to generate the hemisuccinate (Table 2, DAVS), and (3) reacting with isobutyl chloroformate to form the reactive mixed anhydride.

Cryptophycins: Cryptophycin is a naturally occurring, highly potent tubulin inhibitor. Extensive medicinal chemistry efforts to improve potency and manufacturability yielded cryptophycin-52 (LY355703). Most sites on the cyclic depsipeptide cannot be modified without significantly reducing biological activity. Modifications to the C3′-phenyl ring are readily tolerated, however, indicating this site is a handle for the formation of functional conjugates. Synthesis of an amine-bearing derivative of Cryptophycin-52 has been previously described (Eggen and Georg, Medicinal Research Reviews, 22(2):85-101, 2002). This derivative (Table 2, Cryp-NH2) is directly suitable for conjugation.

Tubulysins: Tubulysins are a recently discovered class of highly potent tubulin inhibitors. As linear peptides of modified amino acids, they are amenable to chemical synthesis and conjugation using relatively standard peptide chemistries (e.g. in situ carboxylate activation via carbodiimides). A representative tubulysin structure is shown in Table 2.

Others: A number of other highly potent cytotoxic agents have been identified and characterized, many of which may additionally be suitable for the formation of aptamer-linker-drug conjugates. These would include modified variants ofdolastatin-10, dolastatin-15, auristatin E, rhizoxin, epothilone B, epothilone D, taxoids.

TABLE 2
Cytotoxins For Use in Conjugation with Aptamers

| | |
|---|---|
| Calicheamicins | 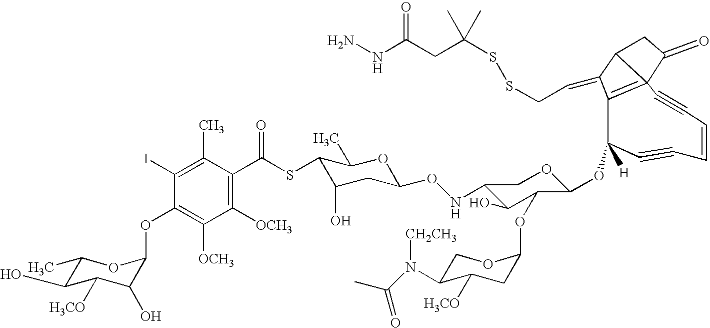 |
| | NAc-gamma calicheamicin dimethyl hydrazide |
| | (NAc-γ-DMH) |
| | 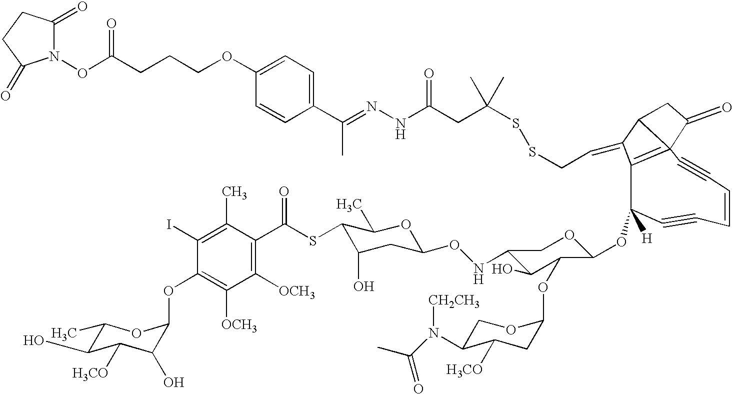 |
| | NAc-gamma calicheamicin-’AcBut'-N-hydroxysuccinimide |
| | (NAc-γ-NHS) |
| Maytansinoids | 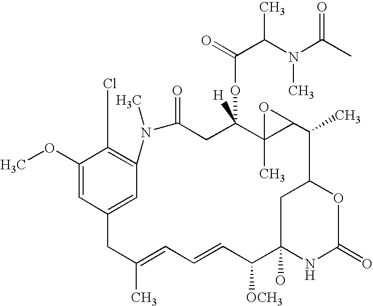 |
| | Maytansine |
| 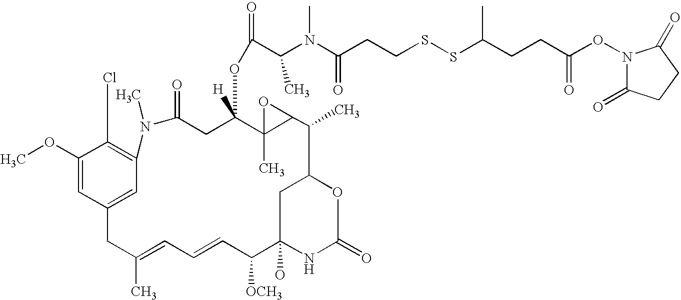 | |
| | May-NHS |
| Vinca alkaloids | 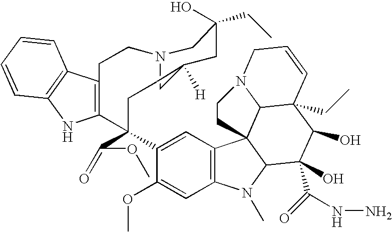 |
| | Desacetyl vinblastine 3-carboxhydrazide (DAVCH) |
| | 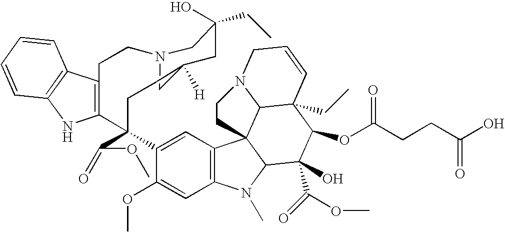 |
| | Desacetyl vinblastine 4-O-succinate (DAVS) |
| Cryptophycins | 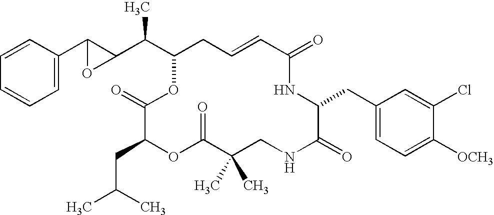 |
| | Cryptophycin-52 |
| | 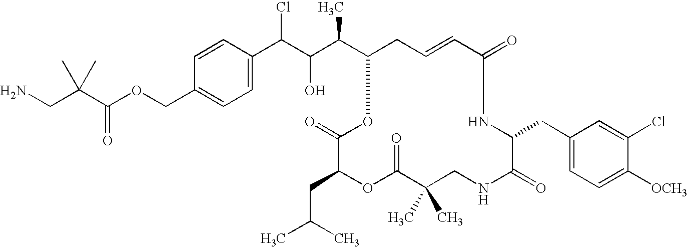 |
| | Cryptophycin-52-amine (Cryp-NH2) |
| Tubulysins | 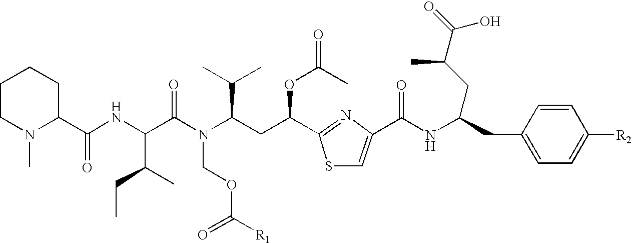 |
| | Representative tubulysin structure (TUB) |

Linkers: The linker portion of the conjugate presents a plurality (i.e., 2 or more) of nucleophilic and/or electrophilic moieties that serve as the reactive attachment points for aptamers and drugs. Nucleophilic moieties include, for example, free amines, hydrazides, or thiols. Electrophilic moieties include, for example, activated carboxylates (e.g. activated esters or mixed anhydrides), activated thiols (e.g. thiopyridines), maleimides, or aldehydes.

To facilitate stepwise synthesis of the conjugate, the reactive attachment points is created or unblocked in situ. For example, a carboxylate-bearing linker is transiently activated by the addition of isobutyl chloroformate to generate a mixed anhydride and subsequently subjected to attack by amine-bearing aptamers and/or drugs. A Boc-protected amine on a heterobifunctional linker (e.g. Boc-amino-PEG-NHS) is deprotected following an initial coupling reaction that quenches its electrophilic moieties. NHS-containing linkers is converted into hydrazide-reactive aldehydes through reaction with mixed amine- and diol-bearing linkers (e.g. aminoglycosides) followed by periodate oxidation. As such, partial reaction of an NHS-containing dendrimer with an amine-bearing aptamer, followed by derivatization with aminoglycoside and oxidation generates a multivalent aldehyde for conjugation.

By using a high molecular weight linker, renal clearance of the conjugate can be minimized, even in the eventuality that aptamers connected to the conjugate are removed (e.g. as a result of nuclease degradation in vivo). Preventing renal elimination increases the in vivo half-life of the drug conjugate and also prevents toxic concentrations of drug from accumulating in the kidneys, a particular concern with high potency cytotoxin conjugates. In the preferred embodiment, the bulk of the linker is composed of one or more chains of polyethylene glycol. The overall molecular weight of the conjugate must be greater than 20,000-40,000 Da to effectively block renal clearance. While synthesis of relatively monodisperse, high molecular weight (20,000-30,000 Da) PEG chains is feasible, it is equally feasible to attach multiple medium (2,000-10,000 Da) molecular weight PEG chains to a central core entity (especially given that aptamers attached to the linker contribute substantially to the overall conjugate size). The reactive attachment points for the aptamers and drugs may be introduced either into the core used to anchor the PEG chains or introduced at the free ends of the PEG chains, i.e., well removed from the core.

Several different types of core molecules are used to anchor PEG chain attachment. Examples include simple small molecules bearing multiple nucleophiles or electrophiles (e.g. erythritol, sorbitol, lysine), linear oligomers or polymers (e.g. oligolysine, dextrans), or singly-reactive molecules with the capacity to self assemble into higher order structures (e.g. phospholipids with the capacity to form micelles or liposomes). Representative linkers are listed in Table 3.

TABLE 3
Linkers For Use in Conjugate Formation

| Linker | Structure |
|---|---|
| Boc-NH2- PEG-NHS | 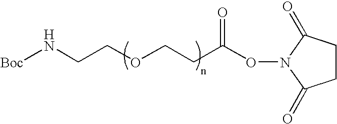 |
| Nucleophilic dendrimers (core =erythritol) | 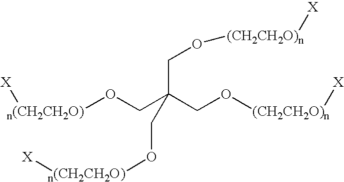 |
| | X = —CH₂CH₂CH₂NH₂ or —CH₂CH₂SH |
| Electrophilic dendrimers (core =erythritol) | 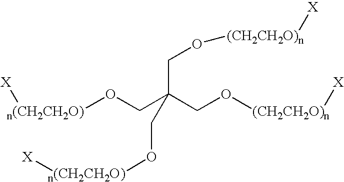 |
| | 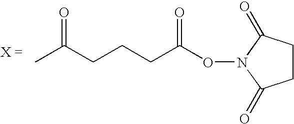 |
| | 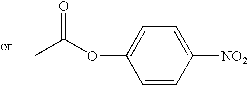 |
| Electrophilic dendrimers (core =octa-polyethylene glycol) | 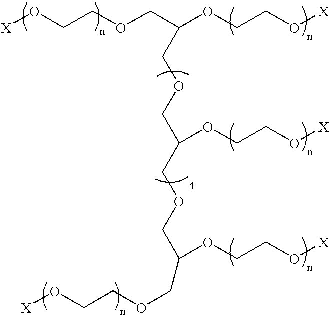 |
| | 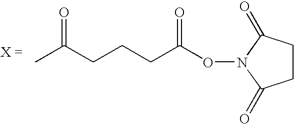 |
| | 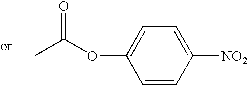 |
| Electrophilic comb polymers | 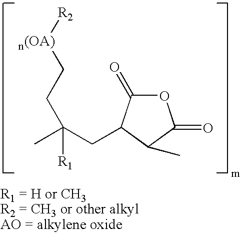 |

Conjugate Synthesis: Table 4 lists examples specific combinations of aptamers, linkers, and drugs that are combined to generate therapeutic aptamer-drug conjugates. In one embodiment, the conjugate synthesis is a one-pot reaction in which aptamer, linker, and drug are combined at appropriate levels to yield the final conjugate. In other embodiments, as noted in Table 4, the stepwise addition of aptamer and drug is required.

In the following table, the term “NH2-aptamer” includes aptamers bearing single and multiple primary amines generated as described above. The term “COOH-aptamer” corresponds to an aptamer bearing a carboxylate at the 5′-terminus as described above. Abbreviations for linkers and drugs correspond to the trivial names provided in tables 2 and 3.

TABLE 4
Methods for Generating Therapeutic Aptamer-Drug Conjugates

| Aptamer | Linker | Drug | Process |
|---|---|---|---|
| Amine | Boc-NH2-PEG-NHS | NAc-γ-NHS | Amine bearing aptamer is reacted with excess Boc-NH2-PEG-NHS |
| Amine | Boc-NH2-PEG-NHS | May-NHS | at 4-20° C. at approximately neutral pH (7-8). The reaction is |
| | | | quenched by the addition of methylamine and the Boc group is |
| | | | removed by reaction with trifluoroacetic acid (TFA) to yield an |
| | | | aptamer-PEG-amine conjugate which is purified by SAX-HPLC. |
| | | | Slight excess of Drug (NAc-γ-NHS or May-NHS) is reacted with the |
| | | | aptamer-PEG-amine at 4-20° C. at approximately neutral pH (7-8). |
| | | | The aptamer-PEG-drug conjugate is isolated by HPLC. |
| Amine | Boc-NH2-PEG-NHS | DAVS | Amine bearing aptamer is reacted with excess Boc-NH2-PEG-NHS |
| Amine | Boc-NH2-PEG-NHS | TUB | at 4-20° C. at approximately neutral pH (7-8). The reaction is |
| | | | quenched by the addition of methylamine and the Boc group is |
| | | | removed by reaction with trifluoroacetic acid (TFA) to yield an |
| | | | aptamer-PEG-amine conjugate which is purified by SAX-HPLC. |
| | | | DAVS or TUB is activated in situ by the addition of triethylamine |
| | | | followed by isobutyl chloroformate to transiently generate the mixed |
| | | | anhydride form of the drug (reaction carried out in dioxane on ice for |
| | | | 1 hour). The pH of the aptamer-PEG conjugate is adjusted to 8.5 by |
| | | | the addition of 1 N NaOH and the conjugate cooled to 5° C. |
| | | | Activated DAVS or TUB is combined with the aptamer conjugate |
| | | | which is stirred at 5° C. for 14 hours, during which time the pH is |
| | | | maintained at 8.5 through addition of 1 N NaOH. |
| Amine | NHS-PEG-erythritol | NAc-γ-NHS | Amine bearing aptamer is reacted with excess NHS-PEG-erythritol |
| Amine | NHS-PEG-erythritol | May-NHS | at 4-20° C. at approximately neutral pH (7-8). The reaction is |
| | | | quenched by the addition of a vast excess of diaminohexane and the |
| | | | aptamer-linker conjugate purified by SAX-HPLC. Slight excess of |
| | | | Drug (NAc-γ-NHS or May-NHS) is reacted with the aptamer-linker |
| | | | conjugate at 4-20° C. at approximately neutral pH (7-8). The |
| | | | resulting conjugate is isolated by HPLC. |
| Amine | NHS-PEG-erythritol | NAc-γ-DMH | Amine bearing aptamer is reacted with excess NHS-PEG-erythritol |
| Amine | NHS-PEG-erythritol | DAVCH | at 4-20° C. at approximately neutral pH (7-8). The reaction is |
| Amine | NHS-PEG-erythritol | Cryp-NH2 | quenched by the addition of excess Drug (Cryp-NH2, NAc-γ-DMH, |
| | | | DAVCH). The resulting conjugate is isolated by HPLC. |
| Amine | pNP-PEG-erythritol | NAc-γ-NHS | Amine bearing aptamer is reacted with excess pNP-PEG-erythritol at |
| Amine | pNP-PEG-erythritol | May-NHS | 4-20° C. at approximately neutral pH (7-8). The reaction is quenched |
| | | | by the addition of a vast excess of diaminohexane and the aptamer- |
| | | | linker conjugate purified by SAX-HPLC. Slight excess of Drug |
| | | | (NAc-γ-NHS or May-NHS) is reacted with the aptamer-linker |
| | | | conjugate at 4-20° C. at approximately neutral pH (7-8). The |
| | | | resulting conjugate is isolated by HPLC. |
| Amine | pNP-PEG-erythritol | NAc-γ-DMH | Amine bearing aptamer is reacted with excess pNP-PEG-erythritol at |
| Amine | pNP-PEG-erythritol | DAVCH | 4-20° C. at approximately neutral pH (7-8). The reaction is quenched |
| Amine | pNP-PEG-erythritol | Cryp-NH2 | by the addition of excess Drug (Cryp-NH2, NAc-γ-DMH, DAVCH). |
| | | | The resulting conjugate is isolated by HPLC. |
| Amine | NHS-PEG-erythritol | DAVS | Amine bearing aptamer is reacted with excess NHS-PEG-erythritol |
| Amine | NHS-PEG-erythritol | TUB | at 4-20° C. at approximately neutral pH (7-8). The reaction is |
| | | | quenched by the addition of a vast excess of diaminohexane and the |
| | | | aptamer-linker conjugate purified by SAX-HPLC. DAVS or TUB is |
| | | | activated in situ by the addition of triethylamine followed by isobutyl |
| | | | chloroformate to transiently generate the mixed anhydride form of |
| | | | the drug (reaction carried out in dioxane on ice for 1 hour). The pH |
| | | | of the aptamer-PEG conjugate is adjusted to 8.5 by the addition of 1 |
| | | | N NaOH and the conjugate cooled to 5° C. Activated DAVS or TUB |
| | | | is combined with the aptamer conjugate which is stirred at 5° C. for |
| | | | 14 hours, during which time the pH is maintained at 8.5 through |
| | | | addition of 1 N NaOH. |
| Amine | pNP-PEG-erythritol | DAVS | Amine bearing aptamer is reacted with excess pNP-PEG-erythritol at |
| Amine | pNP-PEG-erythritol | TUB | 4-20° C. at approximately neutral pH (7-8). The reaction is quenched |
| | | | by the addition of a vast excess of diaminohexane and the aptamer- |
| | | | linker conjugate purified by SAX-HPLC. DAVS or TUB is |
| | | | activated in situ by the addition of triethylamine followed by isobutyl |
| | | | chloroformate to transiently generate the mixed anhydride form of |
| | | | the drug (reaction carried out in dioxane on ice for 1 hour). The pH |
| | | | of the aptamer-PEG conjugate is adjusted to 8.5 by the addition of 1 |
| | | | N NaOH and the conjugate cooled to 5° C. Activated DAVS or TUB |
| | | | is combined with the aptamer conjugate which is stirred at 5° C. for |
| | | | 14 hours, during which time the pH is maintained at 8.5 through |
| | | | addition of 1 N NaOH. |
| Amine | pNP-PEG-octaPEG | NAc-γ-NHS | Amine bearing aptamer is reacted with excess NHS-PEG-octaPEG at |
| Amine | NHS-PEG-octaPEG | May-NHS | 4-20° C. at approximately neutral pH (7-8). The reaction is quenched |
| | | | by the addition of a vast excess of diaminohexane and the aptamer- |
| | | | linker conjugate purified by SAX-HPLC. Slight excess of Drug |
| | | | (NAc-γ-NHS or May-NHS) is reacted with the aptamer-linker |
| | | | conjugate at 4-20° C. at approximately neutral pH (7-8). The |
| | | | resulting conjugate is isolated by HPLC. |
| Amine | NHS-PEG-octaPEG | NAc-γ-DMH | Amine bearing aptamer is reacted with excess NHS-PEG-octaPEG at |
| Amine | NHS-PEG-octaPEG | DAVCH | 4-20° C. at approximately neutral pH (7-8). The reaction is quenched |
| Amine | NHS-PEG-octaPEG | Cryp-NH2 | by the addition of excess Drug (Cryp-NH2, NAc-γ-DMH, DAVCH). |
| | | | The resulting conjugate is isolated by HPLC. |
| Amine | pNP-PEG-octaPEG | NAc-γ-NHS | Amine bearing aptamer is reacted with excess pNP-PEG-octaPEG at |
| Amine | pNP-PEG-octaPEG | May-NHS | 4-20° C. at approximately neutral pH (7-8). The reaction is quenched |
| | | | by the addition of a vast excess of diaminohexane and the aptamer- |
| | | | linker conjugate purified by SAX-HPLC. Slight excess of Drug |
| | | | (NAc-γ-NHS or May-NHS) is reacted with the aptamer-linker |
| | | | conjugate at 4-20° C. at approximately neutral pH (7-8). The |
| | | | resulting conjugate is isolated by HPLC. |
| Amine | pNP-PEG-octaPEG | NAc-γ-DMH | Amine bearing aptamer is reacted with excess pNP-PEG-octaPEG at |
| Amine | pNP-PEG-octaPEG | DAVCH | 4-20° C. at approximately neutral pH (7-8). The reaction is quenched |
| Amine | pNP-PEG-octaPEG | Cryp-NH2 | by the addition of excess Drug (Cryp-NH2, NAc-γ-DMH, DAVCH). |
| | | | The resulting conjugate is isolated by HPLC. |
| Amine | NHS-PEG-octaPEG | DAVS | Amine bearing aptamer is reacted with excess NHS-PEG-octaPEG at |
| Amine | NHS-PEG-octaPEG | TUB | 4-20° C. at approximately neutral pH (7-8). The reaction is quenched |
| | | | by the addition of a vast excess of diaminohexane and the aptamer- |
| | | | linker conjugate purified by SAX-HPLC. DAVS or TUB is |
| | | | activated in situ by the addition of triethylamine followed by isobutyl |
| | | | chloroformate to transiently generate the mixed anhydride form of |
| | | | the drug (reaction carried out in dioxane on ice for 1 hour). The pH |
| | | | of the aptamer-PEG conjugate is adjusted to 8.5 by the addition of 1 |
| | | | N NaOH and the conjugate cooled to 5° C. Activated DAVS or TUB |
| | | | is combined with the aptamer conjugate which is stirred at 5° C. for |
| | | | 14 hours, during which time the pH is maintained at 8.5 through |
| | | | addition of 1 N NaOH. |
| Amine | pNP-PEG-octaPEG | DAVS | Amine bearing aptamer is reacted with excess pNP-PEG-octaPEG at |
| Amine | pNP-PEG-octaPEG | TUB | 4-20° C. at approximately neutral pH (7-8). The reaction is quenched |
| | | | by the addition of a vast excess of diaminohexane and the aptamer- |
| | | | linker conjugate purified by SAX-HPLC. DAVS or TUB is |
| | | | activated in situ by the addition of triethylamine followed by isobutyl |
| | | | chloroformate to transiently generate the mixed anhydride form of |
| | | | the drug (reaction carried out in dioxane on ice for 1 hour). The pH |
| | | | of the aptamer-PEG conjugate is adjusted to 8.5 by the addition of 1 N |
| | | | NaOH and the conjugate cooled to 5° C. Activated DAVS or TUB |
| | | | is combined with the aptamer conjugate which is stirred at 5° C. for |
| | | | 14 hours, during which time the pH is maintained at 8.5 through |
| | | | addition of 1 N NaOH |
| Amine | PEG-comb | NAc-γ-DMH | Amine bearing aptamer is reacted with excess PEG-comb at 4-20° C. |
| Amine | PEG-comb | Cryp-NH2 | at approximately neutral pH (7-8). Excess Drug (NAc-γ-DMH, |
| Amine | PEG-comb | DAVCH | Cryp-NH2, or DAVCH) is added to the aptamer-PEG-comb reaction |
| | | | at 4-20° C. at approximately neutral pH (7-8). The resulting |
| | | | conjugate is isolated by HPLC. |
| COOH | NH2-PEG-erythritol | NAc-γ-NHS | Carboxylate-bearing aptamer is reacted with a slight excess of NH2- |
| COOH | NH2-PEG-erythritol | May-NHS | PEG-erythritol in the presence of EDC at 4-20° C. at pH 4.5-6. The |
| | | | resulting aptamer-linker conjugate purified by SAX-HPLC and |
| | | | reacted with excess Drug (NAc-γ-NHS or May-NHS). The aptamer- |
| | | | linker-drug conjugate is purified by SAX-HPLC. |
| COOH | NH2-PEG-erythritol | DAVS | (1) Stepwise: Carboxylate-bearing aptamer is reacted with a |
| COOH | NH2-PEG-erythritol | TUB | slight excess of NH2-PEG-erythritol in the presence of EDC at 4-20° C. |
| | | | at pH 4.5-6. The resulting aptamer-linker conjugate purified |
| | | | by SAX-HPLC. Drug (DAVS or TUB) is activated in situ by the |
| | | | addition of triethylamine followed by isobutyl chloroformate to |
| | | | transiently generate the mixed anhydride form of the drug |
| | | | (reaction carried out in dioxane on ice for 1 hour). The pH of the |
| | | | aptamer-PEG conjugate is adjusted to 8.5 by the addition of 1 N |
| | | | NaOH and the conjugate cooled to 5° C. Activated DAVS or |
| | | | TUB is combined with the aptamer conjugate which is stirred at |
| | | | 5° C. for 14 hours, during which time the pH is maintained at 8.5 |
| | | | through addition of 1 N NaOH. The aptamer-linker-drug |
| | | | conjugate is purified by SAX-HPLC. |
| | | | One-pot: Carboxylate-bearing aptamer and Drug (DAVS or TUB) |
| | | | at a suitable ratio to achieve the desired loading is reacted with |
| | | | limiting NH2-PEG-erythritol in the presence of EDC at 4-20° C. at |
| | | | pH 4.5-6. The resulting aptamer-linker conjugate purified by |
| | | | SAX-HPLC. |
| Amine | NHS-PEG-erythritol | NAc-γ-DMH | Amine bearing aptamer is reacted with excess NHS-PEG-erythritol |
| Amine | NHS-PEG-erythritol | DAVCH | at 4-20° C. at approximately neutral pH (7-8). The reaction is |
| | | | quenched by the addition of a vast excess of a suitable |
| | | | aminoglycoside and the aptamer-linker conjugate purified by SAX- |
| | | | HPLC. Further reaction with an excess of sodium metaperiodate at |
| | | | 4-20° C. and pH 5.5-6 yields a multivalent aldehyde which can be |
| | | | isolated by size-exclusion chromatography. Excess Drug (NAc-γ- |
| | | | DMH or DAVCH) is added and reacted at 4-20° C. at pH 5.5-7. The |
| | | | resulting conjugate is isolated by HPLC. |
| Amine | NHS-PEG-octaPEG | NAc-γ-DMH | Amine bearing aptamer is reacted with excess NHS-PEG-octaPEG |
| Amine | NHS-PEG-octaPEG | DAVCH | at 4-20° C. at approximately neutral pH (7-8). The reaction is |
| | | | quenched by the addition of a vast excess of a suitable |
| | | | aminoglycoside and the aptamer-linker conjugate purified by SAX- |
| | | | HPLC. Further reaction with an excess of sodium metaperiodate at |
| | | | 4-20° C. and pH 5.5-6 yields a multivalent aldehyde which can be |
| | | | isolated by size-exclusion chromatography. Excess Drug (NAc-γ- |
| | | | DMH or DAVCH) is added and reacted at 4-20° C. at pH 5.5-7. The |
| | | | resulting conjugate is isolated by HPLC. |

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

What is claimed is:

1. An aptamer-toxin conjugate therapeutic agent comprising a targeting moiety conjugated to a cytotoxic moiety.

2. The therapeutic agent of claim 1 wherein said targeting moiety is an aptamer.

3. The therapeutic agent of claim 1 wherein said targeting moiety is a nucleic acid sensor molecule.

4. The therapeutic agent of claim 2 wherein said cytotoxic moiety is selected from the group consisting of a cytotoxic peptide, a cytotoxic protein, a small molecule chemotherapeutic agent, and a radioisotope therapeutic molecule.

5. The therapeutic agent of claim 3 wherein said cytotoxic moiety is selected from the group consisting of a cytotoxic peptide, a cytotoxic protein, a small molecule chemotherapeutic agent, and a radioisotope therapeutic molecule.

6. The therapeutic agent of claim 4, wherein said targeting moiety is conjugated to said cytotoxic moiety by a covalent bond.

7. The therapeutic agent of claim 5, wherein said targeting moiety is conjugated to said cytotoxic moiety by a covalent bond.

8. The therapeutic agent of claim 4 wherein said targeting moiety is conjugated to said cytotoxic moiety by a non-covalent bond.

9. The therapeutic agent of claim 5 wherein said targeting moiety is conjugated to said cytotoxic moiety by a non-covalent bond.

10. An aptamer-drug conjugate comprising one or more aptamers and a drug linked by a linker and having the formula: (aptamer)ₙ—linker—(drug)ₘ, wherein n is between 1 and 10 and m is between 0 and 20.

11. The aptamer-drug conjugate of claim 10, wherein at least one of the one or more aptamers is a tumor-cell targeting aptamer.

12. The aptamer-drug conjugate of claim 10, wherein at least one of the one or more aptamers is specific for a target selected from the group consisting of PSMA, PSCA, e-selectin, an ephrin, ephB2, cripto-1, TENB2 (TEMFF2), ERBB2 receptor (HER2), MUC1, CD44v6, CD6, CD19, CD20, CD22, CD23, CD25, CD30, CD33, CD56, IL-2 receptor, HLA-DR10P subunit, EGFRvIII, MN antigen, caveolin-1 and nucleolin.

13. The aptamer-drug conjugate of claim 10, wherein the drug is a cytotoxin.

14. The aptamer-drug conjugate of claim 10, wherein the drug is selected from the group consisting of a calicheamicin, a maytansinoid, a vinca alkaloid, a cryptophycin, a tubulysin, dolastatin-10, dolastatin-15, auristatin E, rhizoxin, epothilone B, epithilone D, taxoids and variants thereof.

15. The aptamer-drug conjugate of claim 10, wherein the drug is selected from the group consisting of Nac-γ-DMH, Nac-γ-NHS, maytansine, May-NHS, desacetyl vinblastine 3-carboxhydrazide (DAVCH), desacetyl vinblastine 4-O-succinate (DAVS), cryptophycin-52, and crypthophycin-52-amine (Cryp-NH2).

16. The aptamer-drug conjugate of claim 10, wherein the linker comprises one or more nucleophilic moieties, one or more electrophilic moieties or combinations thereof.

17. The aptamer-drug conjugate of claim 10, wherein the linker is selected from the group consisting of a Boc-protected amine, a Boc-protected amine on a heterobifunctional linker, a nucleophilic dendrimer, an electrophilic dendrimer and an electrophilic comb polymer.

18. The aptamer-drug conjugate of claim 10, wherein the linker is selected from the group consisting of Boc-NH2-PEG-NHS, an erythritol dendrimer, an octa-polyethylene glycol dendrimer and comb polymer.
